(19) United States
(12) Patent Application Publication
(10) Pub. No.: US 2017/0138946 A1
(43) Pub. Date: May 18, 2017

(54) BIOMARKERS FOR RESPONSE TO EZH2 INHIBITORS

(71) Applicant: Memorial Sloan-Kettering Cancer Center, New York, NY (US)

(72) Inventors: ROSS LEVINE, New York, NY (US); Lindsay LaFave, New York, NY (US); Omar Abdel-Wahab, New York, NY (US)

(21) Appl. No.: 15/380,807

(22) Filed: Dec. 15, 2016

Related U.S. Application Data
(63) Continuation of application No. PCT/US2015/036677, filed on Jun. 19, 2015.
(60) Provisional application No. 62/014,594, filed on Jun. 19, 2014.

(51) Int. Cl. G01N 33/574; A61K 31/5377; C12Q 1/68

(52) U.S. Cl. CPC G01N 33/5748; C12Q 1/6886; A61K 31/5377; C12Q 2600/106; C12Q 2600/158; G01N 2333/948

(57) ABSTRACT

The presently disclosed subject matter relates to the use of one or more biomarkers to evaluate the likelihood that an EZH2 inhibitor would produce an anti-cancer effect in a subject. It is based, at least in part, on the discovery that loss of BAP1 results in the upregulation of EZH2 expression and activity. In a specific non-limiting embodiment, the method comprises obtaining a sample of the cancer from a subject, and determining, in the sample, the expression level of an BAP1 biomarker, where if the BAP1 biomarker is absent or expressed at lower level in the cancer as compared to a reference control level, then administering a therapeutically effective amount of an EZH2 inhibitor to produce an anti-cancer effect.

### CROSS-REFERENCE TO RELATED APPLICATIONS

This application claims priority to U.S. Provisional Patent Application Ser. No. 62/014,594, filed Jun. 19, 2014, the contents of which is incorporated by reference herein in its entirety.

### GRANT INFORMATION

This invention was made with government support under Grant No. F31CA180642-01 awarded by the National Institutes of Health. The government has certain rights in the invention.

### 1. INTRODUCTION

This present invention relates to biomarkers that may be used to evaluate the likelihood that an EZH2 inhibitor would produce an anti-cancer effect in a subject. As such, these biomarkers may be used in methods of treating cancer patients.

### 2. BACKGROUND OF THE INVENTION

BRCA1 associated protein-1 (BAP1) is an ubiquitin carboxy-terminal hydrolase that is involved in the removal of ubiquitin from proteins. BAP1 binds to the breast cancer type 1 susceptibility protein (BRCA1) via the RING finger domain of BRCA1 and can act as a tumor suppressor. BAP1 is involved in the regulation of transcription, regulation of cell cycle and growth, response to DNA damage and chromatin dynamics. Genome-sequencing studies have shown that germline mutations in BAP1 can be associated with tumor predisposition syndrome (TPDS), which involves increased risk of cancers including malignant mesothelioma, uveal melanoma and cutaneous melanoma. Further studies have identified germline BAP1 mutations associated with other cancers including lung adenocarcinoma and renal cell carcinoma. The prognosis of some patients with BAP1-mutations is quite poor with no identified effective treatments, as many patients with malignant mesothelioma will die from their disease. BAP1 mutations in patients with renal cell carcinoma predict for poor prognosis and BAP1 mutations in uveal melanoma patients predict for a higher risk group and metastasis.

Enhancer of zeste homolog 2 (EZH2), is a member of the Polycomb-group (PcG) family, members of which are involved in the regulation of the transcriptional state of genes by methylation of histone proteins. Drugs have been developed that specifically target EZH2 and the effect of such drugs in patients with a variety of tumors has been an active area of investigation. EZH2 inhibitors are currently being tested clinically in lymphoma patients with EZH2-activating mutations. Accordingly, there is a need in the art for treatments for patients with BAP1-mutations and biomarkers that will be useful to determine when an EZH2 inhibitor should be used to treat a cancer.

### 3. SUMMARY OF THE INVENTION

The present invention relates to the use of one or more biomarkers to evaluate the likelihood that an EZH2 inhibitor would produce an anti-cancer effect in a subject. It is based, at least in part, on the discovery that the loss of BAP1 activity results in the upregulation of EZH2 expression and activity.

Accordingly, in non-limiting embodiments, the present invention provides for assay methods and kits for determining the presence of one or more biomarkers, e.g., BAP1 biomarkers, in a sample from a patient, and methods of using such determinations in selecting a therapeutic regimen for a cancer patient and in methods of treating cancer patients.

The present invention provides for a method for determining whether an anti-cancer effect is likely to be produced in a cancer by an EZH2 inhibitor. In a non-limiting embodiment, the method comprises determining the expression of a BAP1 biomarker in one or more cells of the cancer, where if the BAP1 biomarker is absent or expressed at lower levels in the cancer, as compared to a reference control level, a therapeutically effective amount of an EZH2 inhibitor is administered to produce an anti-cancer effect. In certain non-limiting embodiments, the expression of the BAP1 biomarker can be determined by immunofluorescence, Western Blot, in situ hybridization or polymerase chain reaction. In certain embodiments, the method can further include determining the expression level of EZH2, SUZ12 and/or L3MBTL2 in the sample.

The present invention further provides for a method for treating a subject having a cancer. In certain non-limiting embodiments, the method comprises obtaining a sample of the cancer from the subject, and determining, in the sample, the expression level of a BAP1 biomarker and/or the expression level of EZH2 and/or SUZ12, where if the BAP1 biomarker is absent or expressed at a lower level than a BAP1 reference control level and/or if the expression of EZH2 and/or SUZ12 is increased compared to an EZH2 and/or SUZ12 reference control level, then treatment of the subject with a therapeutically effective amount of an EZH2 inhibitor is initiated.

The present invention further provides for a method for determining whether an anti-cancer effect is likely to be produced in a cancer by an EZH2 inhibitor. In a non-limiting embodiment, the method comprises obtaining a sample of the cancer from a subject, and determining, in the sample, the expression level of an BAP1 biomarker, where if the BAP1 biomarker is absent or expressed at lower level in the cancer, as compared to a reference control level, it is more likely that the EZH2 inhibitor would have an anti-cancer effect on the cancer. In certain embodiments, the BAP1 biomarker is a BAP1 protein biomarker. In certain embodiments, the BAP1 biomarker is a BAP1 nucleic acid biomarker.

The present invention further provides for a method of predicting the sensitivity of a cancer in a patient to an EZH2 inhibitor. In a non-limiting embodiment, the method comprises obtaining a sample of the cancer from the patient and determining the expression level of a BAP1 protein biomarker in the cells comprising the sample, wherein if the BAP1 biomarker is absent or reduced in expression level compared to a reference control level, then the cancer is predicted to be sensitive to an EZH2 inhibitor.

In certain embodiments, the cancer can be malignant mesothelioma, uveal melanoma, renal cell carcinoma, cutaneous melanoma, lung cancer, breast cancer, ovarian cancer, non-melanoma skin cancer, meningioma, cholangiocarcinoma, leomysarcoma, neuroendocrine tumors, pancreatic cancer, paraganglioma, malignant fibrous histiocytoma, melanocytic BAP1-mutated atypical intradermal tumors (MBAITs), acute myeloid leukemia, myelodysplastic syndromes or bladder cancer.

The present invention provides a kit for determining whether an anti-cancer effect is likely to be produced in a cancer by an EZH2 inhibitor. In a non-limiting embodiment, the kit comprises a means for detecting a BAP1 biomarker. In certain embodiments, the means for detecting a BAP1 biomarker comprises one or more packaged primers, probes, arrays/microarrays, biomarker-specific antibodies and/or beads. In certain embodiments, the means for detecting a BAP1 biomarker comprises one or more antibodies, or antigen binding fragment thereof, for detecting a BAP1 biomarker. In certain embodiments, the kit further comprises one or more primers, probe, arrays/microarray, biomarker-specific antibody and/or bead for detecting EZH2 and/or SUZ12 expression.

### 4. BRIEF DESCRIPTION OF FIGURES

FIG. 1. BAP1 loss upregulated histone H3K27me3 In vitro.

FIG. 2. EZH2 was overexpressed in BAP1-mutant mesothelioma cells.

FIG. 3. Gain/loss of BAP1 expression resulted in altered PRC2 subunit expression.

FIG. 4. Inhibition of EZH2 decreased tumor volume in mesothelioma xenografts.

FIG. 5A-L. Characterization of conditional hematopoietic deletion of Bap1. (a) Average gene expression of BAP1, ASXL1, ASXL2, and ASXL3 in TCGA AML (acute myeloid leukemia) and (b) mesothelioma patients as expressed as a mathematical mean with standard error of normalized read counts. (c) Bap1 expression by qRT-PCR in purified populations of hematopoietic cells in C57/B6H mice. LT-HSC, long term hematopoietic stem cells (HSCs), (Lin⁻Sca-1⁺c-Kit⁺CD150⁺CD48⁻); ST-HSC, short term HSCs (Lin⁻Sca-1⁺c-Kit⁺CD150⁺CD48⁺); MPP, mulitpotent progenitor, (Lin⁻ Sca-1⁺c-Kit⁺ CD150⁻CD48⁺); LSK, Lin⁻Sca-1⁺c-Kit⁺; MP, myeloid progentiors (Lin⁻Sca-1⁻c-Kit⁻), GMP, Granulocyte Macrophage Progenitors (Lin⁻Sca-1⁻c-Kit⁺ CD34⁺Fcγ⁺); CMP, Common Mycloid Progenitors (Lin⁻Sca-1⁻c-Kit⁺ CD34⁺Fcγ^{lo}); MEP; Macrophage Erythroid Progenitors (Lin⁻Sca-1⁻c-Kit⁺CD34⁻Fcγ⁻), MONO; monocytes (Mac1⁺Gr1⁻), PMN; (polymorphonuclear neutrophil, Mac1⁺Gr1⁺), T cells, CD3⁺; and B cells, B220⁺. (d) Bap1 targeting scheme in murine embryonic stem cells obtained from the EUCOMM consortium. After chimera generation, mice were crossed with transgenic FLPE mice to excise the premature stop cassette. Mice were then crossed to Mx1-Cre transgenic mice. Genotyping schemes confirming genotype and excision 4 weeks post-polyIpolyC (pIpC) treatment. (e) Enumeration of white blood cells in peripheral blood in control and Bap1 KO mice after treatment with (pIpC) to induce excision and (f) flow cytometric enumeration of myeloid cells (Mac1⁺Gr1⁺). (g) Hematocrit percentages in peripheral blood and (h) flow cytometric enumeration of red blood cell precursors (CD71⁺Ter119⁺) in control and Bap1 KO mouse bone marrow after pIpC-induced excision. (i) Relative frequencies of control and Bap1 KO bone marrow mycloid progenitor populations (Lin⁻c-Kit⁺Sca1⁻). Cells were gated on live lineage-negative populations. (j) Relative quantification of bone marrow myeloid progenitor cell populations (GMP, CMP, MEP) in control and Bap1 KO mice. (k) Flow plots from example control and bone marrow animals to demonstrate progenitor and GMP expansion. (l) Flow cytometric enumeration of cycling progenitor cells (Ki67/DAPI stain); for all experiments: n=5 CON mice and n=8 Bap1 KO mice.

FIG. 6A-L. Bap1 deletion leads to differential pathway activation, as compared to Asxl1 loss, and increased H3K27me3. (a) Spleen images three weeks after conditional deletion of Bap1 and verification of Bap1 deletion by Western blot of control (littermate Bap1f/f mice, CON) and Bap1 knockout (Mx1-Cre Bap1f/f mice, Bap1 KO) bone marrow. (b) Venn diagrams comparing myeloid progenitor gene expression in Bap1 and Asxl1 KO mice, p<0.05; comparisons include gene overlap and genes changing in the same direction. (c) Quantitative real time qPCR (qRT-PCR) of the HoxA cluster in sorted granulocyte-macrophage progenitors (GMPs; Lin⁻c-Kit⁺Sca1⁻CD34⁺ Fcγ⁺); from Bap1 KO and control mice (n=3). (d) Mass spectrometric analysis of purified histones from c-Kit⁺ enriched bone marrow cells from Bap1 KO and controls normalized to total histone H3. (e) Western blot of H3K27me3 and total H3 in purified histones from Bap1 KO and control bone marrow. (f) Number of H3K27me3 broad domains that are called in the CON and Bap1 KO samples. Venn diagram showing unique and overlapping broad domain. (g) Box plot showing normalized H3K27me3 reads in c-Kit enriched bone marrow cells (n=2) (h) Plotting broad domain density as a function of distance from an H3K27me3 domain that was called in both CON and Bap1 KO samples. (i) GSEA demonstrating gene expression correlations to downregulated genes. (j) Local plot of H3K27me3 ChIP-seq at the HOXA locus. (k) Peak calls from H3K27me3 ChIP-Seq in sorted GMP cells displayed in a volcano plot as displayed by ratio (KO/CON) vs. p-value. (l) Significance of specified gene signatures to H3K27me3 bound genes and RNA-Seq. Statistics were calculated with Student t-test; *p<0.05, **p<0.005; ±SEM values reported.

FIG. 7A-C. Bap1 and Asxl1 loss results in opposite gene expression changes. (a) RNA-Seq data of differentially expressed genes in control versus Bap1 KO mice granulocyte-macrophage progenitors (GMPs; Lin⁻c-Kit⁺Sca1⁻CD34⁺ Fcγ⁺); cells analyzed with DESeq2 (cutoff p-value p<0.05). Heatmap indicates genes increasing (red) and decreasing (blue) in expression. (b) Number of positively and negatively enriched genesets from the Bap1 KO and Asxl1 KO GSEA analysis hitting an FDR<0.25 (top). Venn diagram depicting gene sets that are oppositely enriched in Bap1 KO and Asxl1 KO myeloid progenitors by RNA-Seq (bottom). (c) GSEA of oppositely enriched and statistically significant HoxA cluster gene sets in Bap1 KO and Asrl1 KO progenitor cells. p-values and FDR values are indicated.

FIG. 8A-C. Bap1 deletion enhances PRC2 activity. (a) ELISA of H3K27me3 normalized to total H3 in histones purified from bone marrow cells from Bap1 KO and control mice. (b) Percentage of H3K27me3-broad domains called in relation to gene transcriptional start site (promoter, exon, intron, downstream (+/−2 kb), distal (2-5 kb), and intergenic (>50 kb)). (c) Published RNA-Seq from sorted bone marrow populations (Lara-Astiaso et al., 2014) was analyzed and compared to genes that were differentially downregulated and marked with H3K27me3 following Bap1 loss were analyzed using GSEA. Genes that were downregulated and marked by H3K27me3 were only correlated with the hematopoietic progenitor populations, suggesting that these may be the relevant target populations. These data are explanatory of the progenitor expansion that were seen in the Bap1 KO mouse model.

FIG. 9A-C. In vitro BAP1 perturbations lead to changes in H3K27me3. (a) Western blot of SET2 cells transduced with two independent BAP1 shRNAs revealing H3K27me3 levels in purified histones and BAP1 knockdown from whole cell extract. (b) Methylcellulose assay with control and Bap1 KO bone marrow cells. BAP1 cDNA constructs were reintroduced into control and Bap1 deleted cells. Histone ELISA assays were performed for H3K27me3. Quantitative qPCR to assess expression of BAP1 construct. (c) Reintroduction of BAP1 and deubiquitinase mutant BAP1 C91A in Bap1-deficient murine cells. Histone Western blots were performed for H3K27me3 and total H3. Quantitative qPCR to show levels of construct expression.

FIG. 10A-D. Characterization of Bap1/Ezh2 compound KO mice. (a) Bone marrow pathology for various Bap1/Ezh2 genotypes. (b) Flow cytometric staining for erythroid cells in indicated genotypes (CD71, Ter119) and quantification. (I-IV) are indicative of stages of erythroid differentiation with I being the most immature and IV being the most mature. Quantitation of these phenotypes on the right of the representative flow plots. (c) Spleen sizes for indicated genotypes, 4 weeks post-pIpC. (d) White blood cell counts for indicated genotypes, 4 weeks post-pIpC.

FIG. 11A-K. Proliferation induced by Bap1 deletion is rescued by loss of Ezh2. (a) Western blot of H3K27me3 levels in histones purified from bone marrow of Bap1 KO, Ezh2 KO, Bap1/Ezh2 KO and control mice. (b) Representative images of spleens and (c) enumeration of spleen weight from the indicated genotypes of mice, 3 weeks post pIpC. (d) Peripheral white blood cell counts and (e) hematocrit percentages as quantified by a Hemavet. (f) Flow cytometric enumeration of myeloid progenitors (Lin⁻ c-Kit⁺Sca1⁻) and (g) Mature myeloid cells (Mac1⁺Gr1⁺) (h) Cell cycle analyses in myeloid progenitors using Ki67 and DAPI stain (n=3/group) (i) Western blot for H3K27me3 levels in mice (n=5/group) treated with EPZ011989 twice a day at 500 mg/kg for 16 days. (j) Spleen weights and (k) white blood cell counts after treatment. Unless otherwise indicated, n=S/CON, n=5/Ezh2 KO, n=8/Bap1 KO, and n=11/Bap1/Ezh2 KO, Statistics were calculated with Student t-test; *p<0.05, **p<0.005; ±SEM values reported.

FIG. 12A-C. Histone analyses in Bap1 KO animals. (a) EZH2 transcription as assessed by qPCR in control and Bap1 KO cells. Cells were either transduced with empty vector or a BAP1 overexpression construct. (b) Histone mass spectrometry in control and Bap1 KO animals c-kit enriched bone marrow cells, n=2. (c) H4K20mel ChIP-qPCR experiments in 293T cells that overexpress FLAG-tagged BAP1, ASXL1 and Bmi1.

FIG. 13A-L. BAP1 depletion leads to an increase in PRC2 component expression, increased H4K20me1 and deubiquitination of L3MBTL2. (a) Co-immunoprecipitation of endogenous EZH2 and BAP1 in SET2 cells followed by Western blot analysis (performed in presence of benzonase to inhibit interactions dependent on DNA). (b) Bap1, Ezh2 and Suz12 expression by qRT-PCR from sorted granulocyte-macrophage progenitor (GMP; Lin⁻ c-Kit⁺Sca1⁻CD34′Fcγ⁺). (c) Western blot analysis of Bap1 and Ezh2 in bone marrow cells from Bap1 KO and control mice. (d) H4K20me1 quantification from histone mass spectrometry experiments. (e) Cell viability as assessed by Cell Titer Glo viability assay and (f) Annexin V assays for SETD8 overexpression experiments in BAP1 wild-type (MSTO-211H, Meso10) and mutant cell lines H226, deletion; H2452 catalytic mutation). (g) Quantitative qPCR for SETD8 and EZH2 in BAP1 mutant cells with SETD8 overexpression. (h) Western blot analysis for SETD8 and EZH2 in a BAP1 wild-type cell line. (i) Cell Titer Glo assay in cells treated with DMSO or 5, 10, 20 μM BVT594. (j) L3MBTL2 and BAP1 expression in BAP1 wild-type (Met5a, JMN) and mutant mesothelioma cell lines (H226, H2452, H28). (k) 293T cells overexpressing Myc-His tagged ubiquitin and L3MBTL2 cDNA and varying levels of BAP1 (0, 5 μg, 2.5 μg, 1 μg). Co-immunoprecipitation experiments were conducted with Ni-beads and a series of stringent washes. (l) Model depicting the regulation of BAP1 leading to effects on chromatin and gene expression. Statistics were calculated with Student t-test; *p<0.05, **p<0.005; ±SEM values reported.

FIG. 14A-B. Analysis of BAP1, ASXL1, HCF-1, and OGT binding. (a) K-means clustering analyses for BAP1, ASXL1, HCF-1, and OGT ChIP-Seq. (b) Homer de novo motif analyses in BAP1-bound clusters.

FIG. 15A-I. L3MBTL2 and BAP1 co-regulate EZH2. (a) Expression of Bap1 and L3mblt2 in control and Bap1 KO bone marrow cells. (b) Expression of L3mbtl2 by qPCR in GMPs. (c) Western blot of H226 and H2452 cells treated with 25 uM MG132. Insoluble fractions were extracted using 2% SDS containing lysis buffer. (d) Expression of EZH2 in cell lines overexpressing L3MBTL2. (e) EZH2 promoter activity assay with a construct containing 1.9 kB of the EZH2 promoter and a Renilla control vector transiently transfected into 293T cells with either empty vector, a BAP1 or L3MBTL2 expression vector. Firefly luciferase activity was normalized to Renilla activity in each of these conditions. (f) Two independent hairpins were used to knockdown L3MBTL2 protein in SET2 cells. Western blot analyses were conducted on L3MBTL2, EZH2, and actin including short and long exposures. (g) Chip for L3MBTL2 followed by qPCR at the EZH2, SUZ12, E2F6 (positive control), PHF20 (positive control), and MORC3 (negative control) loci in 293T cells. (h) Anti-FLAG ChIP followed by qPCR at the EZH2 locus in 293T cells overexpressing FLAG-L3MBTL2 or FLAG-BAP1. JAM2 is a positive control. Compared to 293T cells without FLAG overexpression. (i) Western blot for L3MBTL2 and BAP1 following respective IPs in 293T cells. Agarose DNA gel included to show DNA digestion.

FIG. 16A-H. BAP1-mutant mesothelioma cell lines and xenograft models are sensitive to EZH2 inhibition. (a) Expression of EZH2 transcripts in TCGA mesothelioma patients compared to matched normals. (b) Annexin V assays in BAP1 wild-type and mutant cell lines expressing either empty vector or hairpins targeting EZH2. (c) Quantitation of Annexin V experiments in mesothelioma cell lines (d) Tumor size of Meso10 and H226 cell lines expressing EZH2 hairpins implanted into NOD-SCID mice, n=6/group. (e) 2D Cell Titer Glo viability assays after 2 week treatment with EPZ011989 at 1.25 μM. (f) 3D Cell Titer Glo viability assays after 3 weeks EPZ011989 treatment at 1.25 μM. (g) Tumor size formation from BAP1 mutant (MSTO and Meso10) or (h) wild-type cells (H226 and H2452) implanted into NOD-SCID mice and treated with either vehicle or 500 mg/kg BID EPZ011989. Tumors were measured 3× weekly, n=6/group. Target inhibition was assessed by histone western blots in extracted tumors (shown in respective figures). Lung pathology of H2452 cells with vehicle and EPZ1011989 treatment. Arrow indicates bulk metastasized tumor. Statistics were calculated with Student t-test; *p<0.05, **p<0.005; ±SEM values reported.

FIG. 17. PRC2 component expression was increased in sorted populations and in whole bone marrow.

FIG. 18. H3K27me3 was locally and globally increased in BAP1 KO mice. Histone methylation in the BAP1 KO animals was assessed by conducting acid extraction followed by western blot on control and BAP1 KO bone marrow. Chromatin Immunoprecipitation Sequencing (ChIP-Seq) was also completed on cKit enriched bone marrow. Overlaying ChIP-Seq with RNA-Sequencing data demonstrated that genes downregulated in the BAP1 KO mice were increasingly marked with H3K27me3. In BAP1 KO mice, H3K27me3 was observed to be increased at EZH2 target genes such as the HOXA locus.

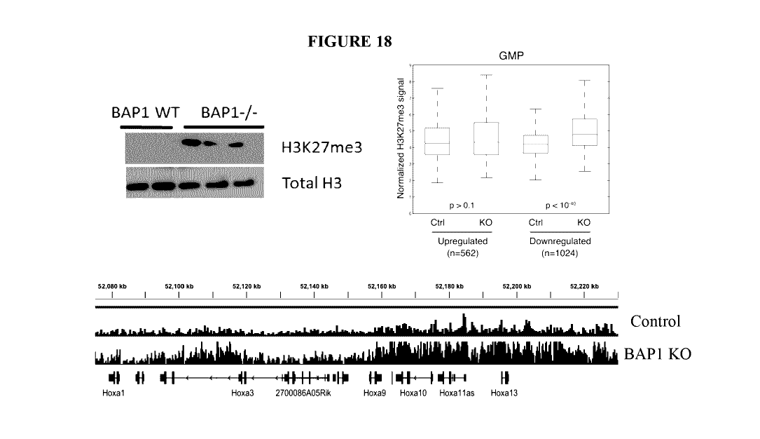

FIG. 19. Upregulation of H3K27me2/3 in BAP1 KO cells occurred at the expense of H3K27me0/l.

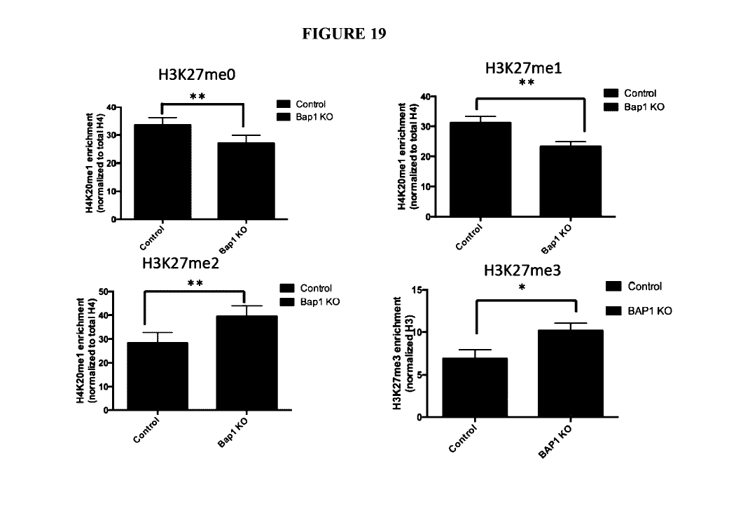

FIG. 20A-D. BAP1 mutant cell lines are most sensitive to EZH2 inhibition. (a) Meso10 cell line overexpressing EZH2 increasingly proliferated after injection into the flank of NOD-SCID mice. (b) EZH2 was overexpressed in MSTO-211H and Meso10 cell lines. The cell lines became increasingly sensitive to EZP011989 with EZH2 overexpression. (c) BAP1-mutant cells became less invasive when treated with the EZH2 inhibitor GSK126. (d) E-Cadherin expression increased in the cell line H226 following treatment with GSK126.

FIG. 21A-C. BAP1-mutant mesothelioma cell lines and xenograft models are sensitive to EZH2 inhibition. (a) Tumor volume of H2452 xenografts treated daily with GSK126 at 150 mg/kg or vehicle (n=10 mice per group). 5 mice from each group were euthanized following 16 days of treatment to assess H3K27me3 depletion. The remaining mice were treated for the remainder of the trial. (b) Histone ELISA and Western blot analysis of H3K27me3 levels in tumors from in vivo treated mice after 16 days treatment. (c) H&E staining, Ki67 staining and TUNEL staining of tumors extracted from vehicle treated and GSK126 treated mice, 10× magnification.

### 5. DETAILED DESCRIPTION

For clarity and not by way of limitation the detailed description of the invention is divided into the following subsections:
(i) BAP1 biomarkers;
(ii) EZH2 inhibitors;
(iii) cancer targets;
(iv) biomarker detection;
(v) methods of use; and
(vi) kits.

### 5.1 Bap1 Biomarkers

The term “biomarker” as used herein, includes nucleic acids and proteins that are related to the activity level of BRCA1 associated protein-1, denoted as BAP1 herein, in a subject.

A “patient” or “subject,” as used interchangeably herein, refers to a human or a non-human subject. Non-limiting examples of non-human subjects include non-human primates, dogs, cats, mice, rats, guinea pigs, rabbits, pigs, fowl, horses, cows, goats, sheep, cetaceans, etc.

In certain non-limiting embodiments, a disclosed BAP1 biomarker may be a nucleic acid. For example, but not by way of limitation, the biomarker can be a deoxyribonucleic acid (DNA) or a ribonucleic acid (RNA), e.g., mRNA.

In certain, non-limiting embodiments, a BAP1 nucleic acid biomarker may be a human BAP1 nucleic acid having the sequence as set forth in NCBI database accession no. NG_031859.1 or NM_004656, or a nucleic acid encoding a BAP1 protein molecule that has the amino acid sequence as set forth in NCBI database accession no. NP_004647.

In a specific, non-limiting embodiment, a BAP1 nucleic acid biomarker may be a mouse BAP1 nucleic acid having the sequence as set forth in NCBI database accession no. NM 027088, or a nucleic acid encoding a BAP1 protein molecule that has the amino acid sequence as set forth in NCBI database accession NP_081364.1.

In a specific, non-limiting embodiment, a BAP1 nucleic acid biomarker may be a rat BAP1 nucleic acid having the sequence as set forth in NCBI database accession no. NM_001107292.1, or a nucleic acid encoding a BAP1 protein molecule that has the amino acid sequence as set forth in NCBI database accession NP 001100762.1.

In certain non-limiting embodiments, a BAP1 biomarker may be a protein.

In a specific, non-limiting embodiment, a BAP1 protein biomarker may be a human BAP1 protein having the amino acid sequence as set forth in NCBI database accession no. NP_004647.

In a specific, non-limiting embodiment, a BAP1 protein biomarker may be a mouse BAP1 protein having the amino acid sequence as set forth in NCBI database accession no. NP 081364.1.

In a specific, non-limiting embodiment, a BAP1 protein biomarker may be a rat BAP1 protein having the amino acid sequence as set forth in NCBI database accession no. NP_001100762.1.

In certain embodiments, the level of the BAP1 biomarker is compared to a reference control level. A “reference control level” or “reference control expression level” of BAP1, as used interchangeably herein, may, for example, be established using a reference control sample. Non-limiting examples of reference control samples include normal and/or healthy cells that have wild-type BAP1 activity. In certain embodiments, a reference control level of BAP1 may, for example, be established using normal cells, e.g., benign cells, located adjacent to the tumor in a patient.

In certain, non-limiting embodiments of the invention, a level of a BAP1 biomarker may be evaluated by evaluating BAP1 function, where the BAP1 expression level is directly proportional to the level of BAP1 function. In one non-limiting example, the function of BAP1 can be downregulation of EZH2 expression (e.g., EZH2 protein expression or nucleic acid expression). For example, and not by way of limitation, the level of a BAP1 biomarker may be determined by determining the expression level of EZH2 in a cancer cell of a subject compared to an EZH2 reference control level. In certain embodiments, an EZH2 reference control level can be established using normal and/or healthy cells that have wild-type and/or normal EZH2 activity and/or normal BAP1 activity. In certain non-limiting embodiments, EZH2 may be a human EZH2 nucleic acid having the sequence as set forth in NCBI database accession no. NG_032043.1; NM_004456.4; NM 001203249.1; NM_152998.2; NM_001203247.1 and/or NM_001203248.1, or a nucleic acid encoding a EZH2 protein molecule that has the amino acid sequence set forth in NCBI database accession no. NP_001190176.1; NP_001190177.1; NP_001190178.1; NP_004447.2; and/or NP_694543.1. In a specific, non-limiting embodiment, EZH2 may be a human EZH2 protein having the amino acid sequence as set forth in NCBI database accession no. NP_001190176.1; NP_001190177.1; NP_001190178.1; NP_004447.2; and/or NP_694543.1.

In one non-limiting example, the function of BAP1 can be regulation of SUZ12 expression (e.g., SUZ12 protein expression or nucleic acid expression). In certain non-limiting embodiments, the level of a BAP1 biomarker may be determined by determining the expression level of SUZ12 in a cancer cell of a subject compared to a SUZ12 reference control level. In certain embodiments, a SUZ12 reference control level can be established using normal and/or healthy cells that have wild-type and/or normal SUZ12 activity and/or normal BAP1 activity. In certain non-limiting embodiments, SUZ12 may be a human SUZ12 nucleic acid having the sequence as set forth in NCBI database accession no. NM_015355.2, or a nucleic acid encoding a SUZ12 protein molecule that has the amino acid sequence set forth in NCBI database accession no. NP_056170.2. In a specific, non-limiting embodiment, SUZ12 may be a human SUZ12 protein having the amino acid sequence as set forth in NCBI database accession no. NP_056170.2.

Where comparisons to reference control expression levels are referred to herein, the biomarker is assessed relative to the reference control expression level within the same species. For example, a human BAP1 biomarker expression level and/or presence are compared with a human BAP1 reference control level.

In particular non-limiting embodiments, the absence and/or a reduced expression of a BAP1 biomarker means the detection of less than about 90%, less than about 80%, less than about 70%, less than about 60%, less than about 50/%, less than about 40%, less than about 30% expression relative to the reference control level.

### 5.2 EZH2 Inhibitors

Non-limiting examples of EZH2 inhibitors include compounds, molecules, chemicals, polypeptides, proteins that inhibit and/or reduce the expression and/or activity of EZH2. Additional non-limiting examples of EZH2 inhibitors include S-adenosyl-methionine-competitive small molecule inhibitors. In particular non-limiting embodiments, the EZH2 inhibitor is derived from tetramethylpiperidinyl compounds. Further non-limiting examples include UNC1999, 3-Deazaneplanocin A (DZNcp), EI1, EPZ-5676, EPZ-6438, GSK343, EPZ005687, EPZ011989 and GSK126.

Further non-limiting examples of EZH2 inhibitors are described in Garapaty-Rao et al., Chemistry and Biology, 20: pp. 1-11 (2013), PCT Patent Application Nos. WO 2013/138361, WO 2013/049770 and WO 2003/070887, and US Patent Application Nos. US 2014/0275081, US 2012/0071418, US 2014/0128393 and US 2011/0251216, the contents of which are hereby incorporated by reference in their entireties.

Further non-limiting examples of EZH2 inhibitors include ribozymes, antisense oligonucleotides, shRNA molecules and siRNA molecules that specifically inhibit the expression or activity of EZH2. One non-limiting example of an EZH2 inhibitor comprises an antisense, shRNA, or siRNA nucleic acid sequence homologous to at least a portion of a EZH2 nucleic acid sequence, wherein the homology of the portion relative to the EZH2 sequence is at least about 75 or at least about 80 or at least about 85 or at least about 90 or at least about 95 or at least about 98 percent, where percent homology can be determined by, for example, BLAST or FASTA software. In certain non-limiting embodiments, the complementary portion may constitute at least 10 nucleotides or at least 5 nucleotides or at least 20 nucleotides or at least 25 nucleotides or at least 30 nucleotides and the antisense nucleic acid, shRNA or siRNA molecules may be up to 15 or up to 20 or up to 25 or up to 30 or up to 35 or up to 40 or up to 45 or up to 50 or up to 75 or up to 100 nucleotides in length. Antisense, shRNA or siRNA molecules may comprise DNA or atypical or non-naturally occurring residues, for example, but not limited to, phosphorothioate residues.

In certain non-limiting embodiments, the EZH2 inhibitor can be used alone or in combination with one or more anti-cancer agents. An anti-cancer agent can be any molecule, compound chemical or composition that has an anti-cancer effect. Anti-cancer agents include, but are not limited to, chemotherapeutic agents, radiotherapeutic agents, cytokines, anti-angiogenic agents, apoptosis-inducing agents or anti-cancer immunotoxins, such as antibodies. “In combination with” means that the EZH2 inhibitor and the one or more anti-cancer agents are administered to a subject as part of a treatment regimen or plan. These terms do not require that the EZH2 inhibitor and one or more anti-cancer agents are physically combined prior to administration nor that they be administered over the same time frame.

### 5.3 Cancer Targets

Non-limiting examples of cancers that may be subject to the presently disclosed subject matter include malignant mesotheliomas, uveal melanomas, renal cell carcinoma, cutaneous melanomas, lung cancer, breast cancer, ovarian cancer, non-melanoma skin cancer, meningioma, chlangiocarcinoma, leiomysarcoma, neuroendocrine tumors, pancreatic cancer, paraganglioma, malignant fibrous histiocytoma, myelodysplastic syndromes, acute myeloid leukemia, melanocytic BAP1-mutated atypical intradermal tumors (MBAITs) and bladder cancer.

### 5.4 Biomarker Detection

Methods for qualitatively and quantitatively detecting and/or determining the expression level of a BAP1 nucleic acid biomarker, an EZH2 nucleic acid, an L3MBTL2 nucleic acid or a SUZ12 nucleic acid include, but are not limited to polymerase chain reaction (PCR), including conventional, qPCR and digital PCR, in situ hybridization (for example, but not limited to Fluorescent In Situ Hybridization (“FISH”)), gel electrophoresis, sequencing and sequence analysis, microarray analysis and other techniques known in the art.

In certain embodiments, the method of detection can be real-time PCR (RT-PCR), quantitative PCR, fluorescent PCR, RT-MSP (RT methylation specific polymerase chain reaction), PicoGreen™ (Molecular Probes, Eugene, Oreg.) detection of DNA, radioimmunoassay or direct radio-labeling of DNA. For example, but not by way of limitation, a nucleic acid biomarker can be reversed transcribed into cDNA followed by polymerase chain reaction (RT-PCR); or, a single enzyme can be used for both steps as described in U.S. Pat. No. 5,322,770, or the biomarker can be reversed transcribed into cDNA followed by symmetric gap ligase chain reaction (RT-AGLCR) as described by R. L. Marshall, et al., PCR Methods and Applications 4: 80-84 (1994). Non-limiting examples of primers for use in the disclosed methods are shown in Table 1.

In certain embodiments, quantitative real-time polymerase chain reaction (qRT-PCR) is used to evaluate mRNA levels of biomarker. The levels of a biomarker and a control mRNA can be quantitated in cancer tissue or cells and adjacent benign tissues. In certain embodiments, the levels of one or more biomarkers can be quantitated in a biological sample.

In a non-limiting embodiment, the method of detection of the present invention can be carried out without relying on amplification, e.g., without generating any copy or duplication of a target sequence, without involvement of any polymerase, or without the need for any thermal cycling. In certain embodiments, detection of the present invention can be performed using the principles set forth in the QuantiGene™ method described in U.S. application Ser. No. 11/471,025, filed Jun. 19, 2006, and is incorporated herein by reference.

In certain embodiments, in situ hybridization visualization can be employed, where a radioactively labeled antisense RNA probe is hybridized with a thin section of a biological sample, e.g., a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

In certain non-limiting embodiments, evaluation of nucleic acid biomarker expression can be performed by fluorescent in situ hybridization (FISH). FISH is a technique that can directly identify a specific region of DNA or RNA in a cell and therefore enables visual determination of the biomarker expression in tissue samples. The FISH method has the advantages of a more objective scoring system and the presence of a built-in internal control consisting of the biomarker gene signals present in all non-neoplastic cells in the same sample. FISH is a direct in situ technique that can be relatively rapid and sensitive, and can also be automated. Immunohistochemistry can be combined with a FISH method when the expression level of the biomarker is difficult to determine by FISH alone.

In certain embodiments, expression of a nucleic acid biomarker can be detected on a DNA array, chip or a microarray. Oligonucleotides corresponding to the biomarker(s) are immobilized on a chip which is then hybridized with labeled nucleic acids of a biological sample, e.g., tumor sample, obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. Patent Application Nos. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See, for example, U.S. Patent Application No. 20030215858).

In certain embodiments, to monitor a nucleic acid biomarker, e.g., BAP1 mRNA, expression levels, mRNA can be extracted from the biological sample to be tested, reverse transcribed and fluorescent-labeled cDNA probes can be generated. The labeled cDNA probes can then be applied to microarrays capable of hybridizing to a biomarker, allowing hybridization of the probe to microarray and scanning the slides to measure fluorescence intensity. This intensity correlates with the hybridization intensity and expression levels of the biomarker.

Types of probes for detection of nucleic acid biomarkers include cDNA, riboprobes, synthetic oligonucleotides and genomic probes. The type of probe used will generally be dictated by the particular situation, such as riboprobes for in situ hybridization, and cDNA for Northern blotting, for example. In certain non-limiting embodiments, the probe is directed to nucleotide regions unique to the particular biomarker RNA. The probes can be as short as is required to differentially recognize the particular biomarker mRNA transcripts, and can be as short as, for example, 15 bases. Probes of at least 17 bases, 18 bases and 20 bases can also be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, ³²P and ³⁵S, or fluorophores. Labeling with radioisotopes can be achieved, whether the probe is synthesized chemically or biologically, by the use of suitably labeled bases.

Methods for detecting and/or determining the level of a protein biomarker, e.g., a BAP1 protein biomarker, EZH2 protein, L3MBTL2 protein or SUZ12 protein, are well known to those skilled in the art, and include, but are not limited to, mass spectrometry techniques, I-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see, for example, U.S. Patent Application Nos. 2003/0013208; 2002/0155493, 2003/0017515 and U.S. Pat. Nos. 6,329,209 and 6,365,418, herein incorporated by reference in their entireties.

In certain non-limiting embodiments, a detection method for measuring protein biomarker expression includes the steps of: contacting a biological sample, e.g., a tissue sample, with an antibody or variant (e.g., fragment) thereof, which selectively binds the biomarker, and detecting whether the antibody or variant thereof is bound to the sample. The method can further include contacting the sample with a second antibody, e.g., a labeled antibody. The method can further include one or more washing steps, e.g., to remove one or more reagents.

In certain non-limiting embodiments, Western blotting can be used for detecting and quantitating biomarker protein expression levels. Cells can be homogenized in lysis buffer to form a lysate and then subjected to SDS-PAGE and blotting to a membrane, such as a nitrocellulose filter. Antibodies (unlabeled) can then brought into contact with the membrane and assayed by a secondary immunological reagent, such as labeled protein A or anti-immunoglobulin (suitable labels including ¹²⁵I, horseradish peroxidase and alkaline phosphatase). Chromatographic detection can also be used. In certain embodiments, immunodetection can be performed with antibody to a biomarker using the enhanced chemiluminescence system (e.g., from PerkinElmer Life Sciences, Boston, Mass.). The membrane can then be stripped and re-blotted with a control antibody specific to a control protein, e.g., actin.

Immunohistochemistry can be used to detect the expression and/or presence of a biomarker, e.g., in a biopsy sample. A suitable antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay can be scored visually, using microscopy, and the results can be quantitated. Machine-based or autoimaging systems can also be used to measure immunostaining results for the biomarker.

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Labeled antibodies against biomarkers can also be used for imaging purposes, for example, to detect the presence of a biomarker in cells of a subject. Suitable labels include radioisotopes, iodine (¹²⁵I, ¹²¹I), carbon (¹⁴C), sulphur (³⁵S), tritium (³H), indium (¹¹²In), and technetium (99mTc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red. The labeled antibody or antibody fragment will preferentially accumulate at the location of cells which contain a biomarker. The labeled antibody or variant thereof, e.g., antibody fragment, can then be detected using known techniques.

Antibodies include any antibody, whether natural or synthetic, full length or a fragment thereof, monoclonal or polyclonal, that binds sufficiently strongly and specifically to the biomarker to be detected. An antibody can have a K_{d} of at most about 10⁻⁶M, 10⁻⁷M, 10⁻⁸M, 10⁻⁹M, 10⁻¹⁰M, 10⁻¹¹M and 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies, and derivatives thereof, that can be used encompass polyclonal or monoclonal antibodies, synthetic and engineered antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof; including, but not limited to, Fv, Fab, Fab′ and F(ab′)₂ fragments, can be used. Such fragments can be produced by enzymatic cleavage or by recombinant techniques. Non-limiting examples of commercially available BAP1 antibodies include SC-8132, SC-48386, SC-13576, SC-28236, SC-8133 and SC-28383 from Santa Cruz Biotechnology (Santa Cruz, Calif.), Ab167250 from Abcam (Cambridge, England) and HPA028814 from Sigma-Aldrich (St. Louis, Mo.). Non-limiting examples of commercially available EZH2 antibodies include Cat Nos. 39933, 39875 and 39901 from Active Motif (Carlsbad, Calif.), 07-689 from Millipore (Billerica, Mass.) and PA1-46476 and PA5-24594 from Thermo Fisher Scientific (Waltham, Mass.). A non-limiting example of a commercially available SUZ12 antibody includes Ab12073 from Abcam. A non-limiting example of a commercially available L3MBTL2 antibody includes 39569 from Active Motif.

In certain non-limiting embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker).

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos. 2003/0199001, 2003/0134304, 2003/0077616, which are herein incorporated by reference in their entireties.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait at al., Science 262:89-92 (1993); Keough at al., Proc. Natl. Acad. Sci. USA. 96:7131-6 (1999); reviewed in Bergman, EXS 88:133-44 (2000).

Detection of the presence of a biomarker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis), to determine the relative amounts of a particular biomarker. Software programs such as the Biomarker Wizard program (Ciphergen Biosystems, Inc., Fremont, Calif.) can be used to aid in analyzing mass spectra.

Additional methods for determining nucleic acid and/or protein biomarker expression in samples are described, for example, in U.S. Pat. No. 6,271,002; U.S. Pat. No. 6,218,122; U.S. Pat. No. 6,218,114; and U.S. Pat. No. 6,004,755; and in Wang et al, J. Clin. Oncol., 22(9): 1564-1671 (2004); and Schena et al, Science, 270:467-470 (1995); all of which are incorporated herein by reference in their entireties.

### 5.5 Methods of Use

In certain non-limiting embodiments, the present invention provides for methods of determining whether an anti-cancer effect is likely to be produced in a cancer by an EZH2 inhibitor, comprising, determining the presence, absence and/or expression level of a BAP1 biomarker, e.g., a BAP1 nucleic acid and/or protein biomarker. Methods for determining the presence, absence and/or expression levels of a BAP1 biomarker are set forth in section 5.4 above. Cancers suitable for treatment are described above in section 5.3. EZH2 inhibitors are described above in section 5.2.

In certain embodiments, the present disclosure provides for a method of producing an anti-cancer effect in a cancer, comprising determining whether cells of the cancer contain a BAP1 biomarker, where if the BAP1 biomarker is absent and/or expressed at lower levels in the cancer, as compared to a reference control level, administering a therapeutically effective amount of an EZH2 inhibitor to produce an anti-cancer effect. Alternatively, if the BAP1 biomarker is found to be expressed at the same or higher levels relative to a reference control level, then an alternative therapy with an agent that is not an EZH2 inhibitor is administered.

A “therapeutically effective amount” refers to an amount that is able to achieve one or more of an anticancer effect, prolongation of survival and/or prolongation of period until relapse.

An “anti-cancer effect” refers to one or more of a reduction in aggregate cancer cell mass, a reduction in cancer cell growth rate, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse or progression-free survival in a patient diagnosed with cancer.

In certain embodiments, the present disclosure provides for a method of producing an anti-cancer effect in a cancer, comprising determining the expression level of a BAP1 biomarker in one or more cells of the cancer, where if the BAP1 protein biomarker is absent and/or expressed at lower levels in the cells, as compared to a reference control level, then administering a therapeutically effective amount of an EZH2 inhibitor to produce an anti-cancer effect. In certain embodiments, the reference control is the level of BAP1 in normal cells, e.g., benign cells, located adjacent to the cancer.

In certain non-limiting embodiments, the level of BAP1 in the cancer sample and/or the reference control sample can be normalized against a nucleic acid and/or protein present in both samples, e.g., a reference protein or nucleic acid such as a housekeeping gene and/or protein, to allow comparison. For example, and not by way of limitation, the reference protein or nucleic acid can be actin or tubulin.

In certain non-limiting embodiments, the present disclosure provides a method of predicting the sensitivity of a cancer in a patient to an EZH2 inhibitor, comprising, obtaining a sample of the cancer from the patient and determining the expression level of a BAP1 protein biomarker in the cells comprising the sample, where if the BAP1 protein biomarker is absent or reduced in expression compared to a reference control level, then the cancer is predicted to be sensitive to the EZH2 inhibitor. In certain embodiments, if the cancer of a patient is predicted to be sensitive to the EZH2 inhibitor, the patient can then be treated with an EZH2 inhibitor. In certain embodiments, if the cancer of a patient is predicted to be insensitive to the EZH2 inhibitor, then the patient can be treated with an agent that is not an EZH2 inhibitor.

In certain non-limiting embodiments, the present disclosure provides a method for treating a subject having a cancer. For example, and not by way of limitation, the method comprises obtaining a sample of the cancer from the subject, and determining, in the sample, the expression level of a BAP1 biomarker, where if the BAP1 biomarker is absent or expressed at a lower level than a BAP1 reference control level, then initiating treatment of the subject with a therapeutically effective amount of an EZH2 inhibitor. Alternatively, if the BAP1 biomarker is found to be expressed at the same or higher levels relative to a reference control level, then the subject may be treated with an agent that is not an EZH2 inhibitor.

In certain embodiments, the methods of the present invention may further comprise detecting the expression level of EZH2, L3MBTL2 and/or SUZ12 in the sample. For example, but not by way of limitation, the mRNA and/or protein expression levels of EZH2, L3MBTL2 and/or SUZ12 can be detected. In certain embodiments, a method for treating a subject having a cancer, comprising, obtaining a sample of the cancer from the subject, and determining, in the sample, the expression level of a BAP1 biomarker and the expression level of EZH2, L3MBTL2 and/or SUZ12, where if the BAP1 biomarker is absent or expressed at a lower level than a BAP1 reference control level and the expression of EZH2 and/or SUZ12 is increased compared to an EZH2 and/or SUZ12 reference control level (and/or the expression of L3MBTL2 is decreased compared to a L3MBTL2 reference control level), then initiating treatment of the subject with a therapeutically effective amount of an EZH2 inhibitor. In certain embodiments, a sample may be collected before and after treatment with an EZH2 inhibitor and the EZH2 expression levels of the samples can be compared.

In certain non-limiting embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, nipple aspirate, saliva, broncho-alveolar lavage, tears and cerebrospinal fluid) and tissue samples. The source of the sample may be solid tissue (e.g., from a fresh, frozen, and/or preserved organ or tumor sample, tissue sample, biopsy, or aspirate), blood or any blood constituents, bodily fluids (such as, e.g., urine, lymph, cerebral spinal fluid, amniotic fluid, peritoneal fluid, or interstitial fluid), or cells from the individual, including circulating tumor cells. In certain non-limiting embodiments, the sample is obtained from a tumor. In certain embodiments, the sample may be a “clinical sample,” which is a sample derived from a patient.

### 5.6 Kits

In certain non-limiting embodiments, the present invention provides for a kit for determining whether an anti-cancer effect is likely to be produced in a cancer by an EZH2 inhibitor, comprising a means for detecting a BAP1 biomarker, as set forth in the preceding sections. Said kit may further include instructions or supporting material that describe the use of the kit to determine whether an anti-cancer effect is likely to be produced in a cancer by an EZH2 inhibitor and/or reference to a website or publication describing same.

Types of kits include, but are not limited to, packaged biomarker-specific probe and primer sets (e.g., TaqMan probe/primer sets), arrays/microarrays, biomarker-specific antibodies, biomarker-specific beads, which further contain one or more probes, primers, or other reagents for detecting one or more biomarkers of the present invention.

In certain non-limiting embodiments, the present invention provides for a kit for determining whether an anti-cancer effect is likely to be produced in a cancer by an EZH2 inhibitor, comprising a means for detecting the presence of a BAP1 nucleic acid biomarker.

In a specific, non-limiting embodiment, a kit may comprise a pair of oligonucleotide primers, suitable for polymerase chain reaction (PCR) or nucleic acid sequencing, for detecting the nucleic acid biomarker(s) to be identified. A pair of primers may comprise nucleotide sequences complementary to a biomarker set forth above, and be of sufficient length to selectively hybridize with said biomarker. Alternatively, the complementary nucleotides may selectively hybridize to a specific region in close enough proximity 5′ and/or 3′ to the biomarker position to perform PCR and/or sequencing. Multiple biomarker-specific primers may be included in the kit to simultaneously detect more than one biomarker. The kit may also comprise one or more polymerases, reverse transcriptase and nucleotide bases, wherein the nucleotide bases can be further detectably labeled.

In certain non-limiting embodiments, a primer may be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length. Non-limiting examples of primers are provided in Table 1. For example, but not by way of limitation, a primer of the present disclosure can comprise one or more of the sequences disclosed in Table 1.

In a further non-limiting embodiment, the oligonucleotide primers may be immobilized on a solid surface, substrate or support, for example, on a nucleic acid microarray, wherein the position of each oligonucleotide primer bound to the solid surface or support is known and identifiable. The oligonucleotides can be affixed to a substrate, such as glass, plastic, paper, nylon or other type of membrane, filter, chip, bead, or any other suitable solid support. The polynucleotides can be synthesized directly on the substrate, or synthesized separate from the substrate and then affixed to the substrate. The arrays are prepared using known methods.

In a specific, non-limiting embodiment, a kit may comprise at least one nucleic acid probe, suitable for in situ hybridization or fluorescent in situ hybridization, for detecting the biomarker(s) to be identified. Such kits will generally comprise one or more oligonucleotide probes that have specificity for various biomarkers. Means for testing multiple biomarkers may optionally be comprised in a single kit.

In certain embodiments, the kits may comprise containers (including microliter plates suitable for use in an automated implementation of the method), each with one or more of the various reagents (typically in concentrated form) utilized in the methods, including, for example, pro-fabricated microarrays, buffers, the appropriate nucleotide triphosphates (e.g., dATP, dCTP, dGTP and dTTP, or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase).

In non-limiting embodiments, the present invention provides for a kit for determining whether an anti-cancer effect is likely to be produced in a cancer by an EZH2 inhibitor, comprising a means for detecting the levels of a BAP1 protein biomarker.

In non-limiting embodiments, a kit may comprise at least one antibody, or antigen-binding fragment thereof, for immunodetection of the biomarker(s) to be identified. Antibodies, both polyclonal and monoclonal, specific for a biomarker, may be prepared using conventional immunization techniques, as will be generally known to those of skill in the art. The immunodetection reagents of the kit may include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (³H, ³⁵S, ³²P, ¹⁴C or ¹³¹I) or enzymes (alkaline phosphatase, horseradish peroxidase).

In a further non-limiting embodiment, the one or more biomarker-specific antibodies may be provided bound to a solid support, such as a column matrix, an array or well of a microtiter plate. Alternatively, the support may be provided as a separate element of the kit.

In certain non-limiting embodiments, where the measurement means in the kit employs an array, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, a kit of the present disclosure may contain one or more probes, primers, antibodies or other detection reagents for detecting the expression level of EZH2 in the sample. For example, a kit can contain an antibody, or fragment thereof; for the detection of protein expression level of EZH2 in a biological sample.

In certain non-limiting embodiments, a kit of the present disclosure may contain one or more probes, primers, antibodies or other detection reagents for detecting the expression level of SUZ12 in the sample. For example, a kit can contain an antibody, or fragment thereof, for the detection of protein expression level of SUZ12 in a biological sample.

In certain non-limiting embodiments, a kit of the present disclosure may contain one or more probes, primers, antibodies or other detection reagents for detecting the expression level of L3MBTL2 in the sample. For example, a kit can contain an antibody, or fragment thereof, for the detection of protein expression level of L3MBTL2 in a biological sample.

A kit may further contain means for allowing comparison between the biomarker level within the cancer sample and the biomarker level in a reference control sample. For example, but not by way of limitation, a kit of the present disclosure may contain one or more probes, primers, antibodies or other detection reagents for detecting a reference protein or mRNA, which can be used to normalize the expression levels of the one or more biomarkers from the samples to allow comparison. Non-limiting examples of a reference protein, e.g., a housekeeping protein, include alpha- or beta-tubulin, actin, cofilin, vinculin and GADPH.

In certain non-limiting embodiments, a kit can further include instructions for using the kit to detect the biomarker of interest. For example, the instructions can describe that the absence and/or a lower expression of a BAP1 biomarker, set forth herein, in a cancer sample from a patient, as compared to a reference control level, is indicative of an increased possibility of producing an anti-cancer effect in the cancer by an EZH2 inhibitor.

In certain embodiments, a kit of the present disclosure can further include one or more EZH2 inhibitors. Non-limiting examples of EZH2 inhibitors are disclosed in section 5.2.

The following examples are offered to more fully illustrate the disclosure, but are not to be construed as limiting the scope thereof.

### 6. EXAMPLE 1: Loss of Bap1 Results in Increased Ezh2 Expression and Activity In Vitro

In this example, the mechanism by which loss of BAP1 activity results in disease states was investigated in vitro.

### 6.1. Results

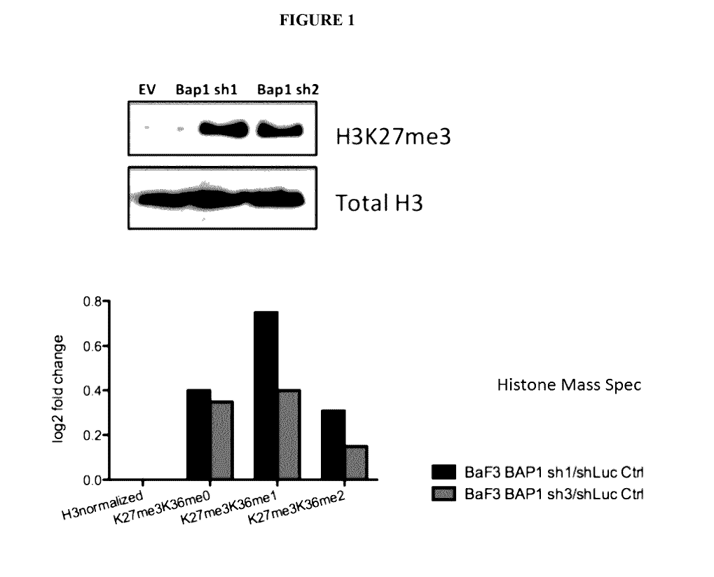

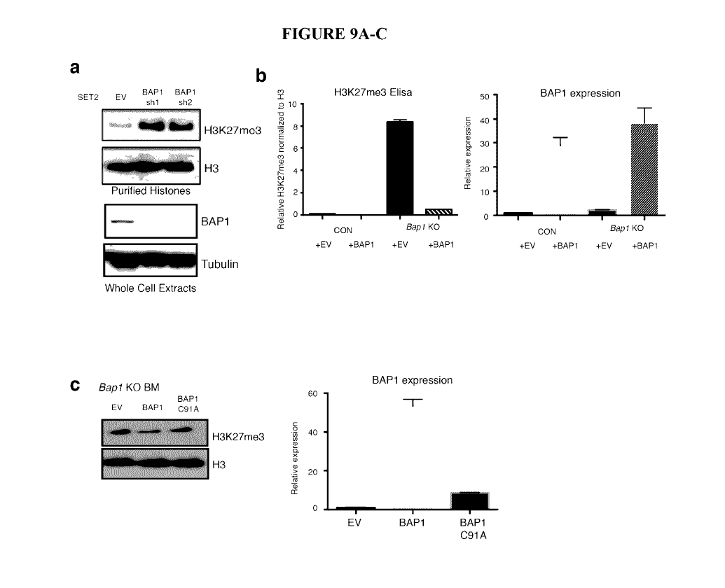

SET2 cells were transduced with shRNA targeting BAP1 to reduce BAP1 expression in vitro. Reduction in BAP1 expression, as validated by western blot, resulted in an increase in the trimethylation of Histone 3 at K27 (H3K27me3) (FIG. 1). See also FIG. 9A. BAP1 protein expression was depleted in BaF3 cells, with confirmation of BAP1 loss by western blot, and histone mass spectrometry was performed. BAP1 knockdown in BaF3 cells revealed an increase in H3K27me3 (FIG. 1).

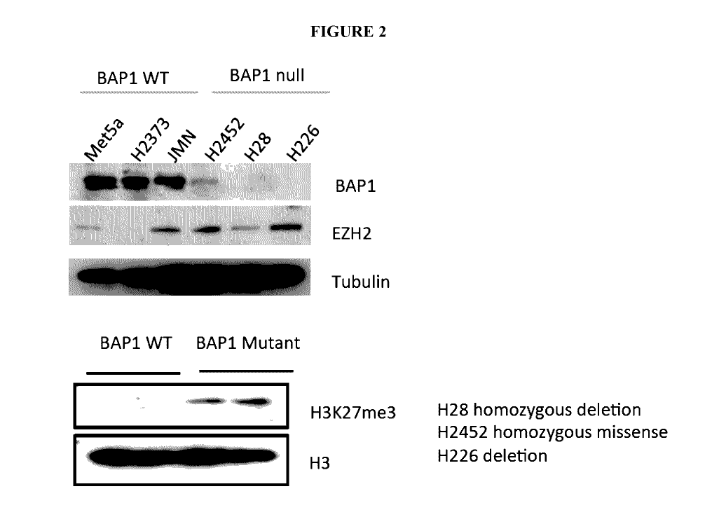

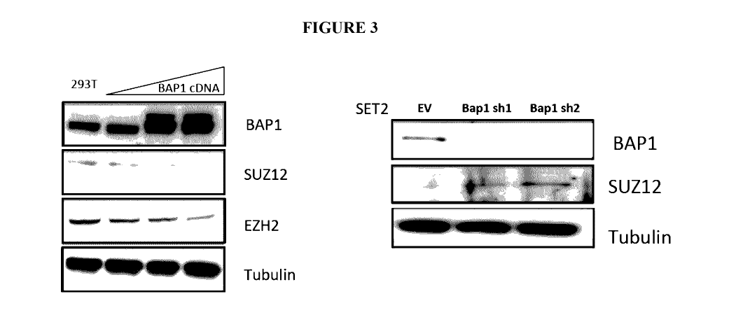

BAP1 has been shown to be highly mutated in solid tumors (Carbone et al., 2012) such as in malignant mesothelioma and uveal melanoma. In mesothelioma cells, which had mutations in BAP1, e.g., H28 homozygous deletion, H2452 homozygous missense and H226 deletion mutations, upregulation of EZH2 expression compared to cells with wild-type BAP1 activity was observed (FIG. 2). Furthermore, in vitro overexpression of BAP1 in 293T cells resulted in the reduction of EZH2 and SUZ12 expression, whereas loss of BAP1 expression resulted in the upregulation of SUZ12 expression (FIG. 3).

### 6.2. Discussion

As described above, the loss of BAP1 protein expression caused an increase in H3K27me3, a repressive chromatin mark placed by EZH2, and an increase in the expression of EZH2 and SUZ12 in in vitro cancer cell line systems. EZH2 inhibitors are currently being tested clinically in lymphoma patients with EZH2-activating mutations. Therefore, BAP1 mutation status could assist in identifying patients that may respond to treatment with EZH2 inhibitors, which may be effective in extending survival in BAP1-mutant patients.

### 7. EXAMPLE 2: Loss of Bap1 Results in Increased Ezh2 Expression and Activity In Vivo

### 7.1 Methods and Materials

Primers:

TABLE 1

| Gene | Genotyping Primers (mouse) |
|---|---|
| Bap1 up | ACTGCAGCAATGTGGATCTG (SEQ ID NO: 1) |
| Bap1 down | GAAAAGGTCTGACCCAGATCA (SEQ ID NO: 2) |
| Bap1 fox F | GCGCAACGCAATTAATGATA (SEQ ID NO: 3) |
| Bap1 fox R | CAGTGTCCAGAATGGCTCAA (SEQ ID NO: 4) |
| Gene | Mutagenesis Primers (human) |
| BAP1 C91A | CCACCAGCTGATACCCAACTCTGCTGCAACTCATGC |
| sense | (SEQ ID NO: 5) |
| BAP1 C91A | GCATGAGTTGCAGCAGAGTTGGGTATCAGCTGGTGG |
| antisense | (SEQ ID NO: 6) |
| Gene | Mouse qPCR Primers |
| Ezh2 F | AGCACAAGTCATCCCGTTAAAG (SEQ ID NO: 7) |
| Ezh2 R | AATTCTGTTGTAAGGGCGACC (SEQ ID NO: 8) |
| Suz12 F | GGCTGACCACGAGCTITTC (SEQ ID NO: 9) |
| Suz12 R | TGGTGCGATAAGATTTCGAGTTC |
| | (SEQ ID NO: 10) |
| Bap1 F | GTTGGTGGATGACACGTCTG (SEQ ID NO: 11) |
| Bap1 R | CTCAGGACTGAAGCCTTTGG (SEQ ID NO: 12) |
| Actin B F | GATCTGGCACCACACCTTCT (SEQ ID NO: 13) |
| Actin B R | CCATCACAATGCCTGTGGTA (SEQ ID NO: 14) |
| HoxA5 F | GCTCAGCCCCAGATCTACC (SEQ ID NO: 15) |
| HoxA5 R | GGCATGAGCTATTTCGATCC (SEQ ID NO: 16) |
| HoxA6 F | CCCTGTTTACCCCTGGATG (SEQ ID NO: 17) |
| HaxA6 R | ACCGACCGGAAGTACACAAG (SEQ ID NO: 18) |
| HoxA8 F | CTTCTCCAGTTCCAGCGTCT (SEQ ID NO: 19) |
| HoxA8 R | AGGTAGCGGTTGAAATGGAA (SEQ ID NO: 20) |
| HoxA9 F | ATGCTTGTGGTTCTCCTCCA (SEQ ID NO: 21) |
| HoxA9 R | GTTCCAGCGTCTGGTGTTTT (SEQ ID NO: 22) |
| Gene | Human qPCR Primers |
| E-CAD F | GACCGGTGCAATCTTCAAA (SEQ ID NO: 23) |
| E-CAD R | TTGACGCCGAGAGCTACAC (SEQ ID NO: 24) |
| HPRT F | CATTATGCCGAGGATTTGG (SEQ ID NO: 25) |
| HPRT R | GCAAGTCTTTCAGTCCTGT (SEQ ID NO: 26) |
| BAP1 F | CGATCCATTTGAACAGGAAGA (SEQ ID NO: 27) |
| BAP1 R | CTCGTGGAAGATTTCGGTGT (SEQ ID NO: 28) |
| Gene | ChIP qPCR Primers (human) |
| EZH2-1 F | AGCTGACTCAAGCTGCTTGT (SEQ ID NO: 29) |
| EZH2-1 R | CAGGAAACCTGAGATTTTCA (SEQ ID NO: 30) |
| EZH2-2 F | CTCAGGACAGTTCTGTTTGG (SEQ ID NO: 31) |
| EZH2-2 R | TCTGACTTAGTTGGAGAACT (SEQ ID NO: 32) |
| SUZ12-1 R | TGAATACAGATGCAGTTATAAGAGAGA |
| | (SEQ ID NO: 33) |
| MORC3 F | CATCTTCCCCAAGCTCCCAAT (SEQ ID NO: 34) |
| MORC3 R | GAGCGAGCTACAAAGCCAGGA (SEQ ID NO: 35) |
| E2F6 F | CCTGTTCCCTTCCTCTGGAA (SEQ ID NO: 36) |
| E2F6 R | CGACGCAGACGGAAAAAGAG (SEQ ID NO: 37) |
| PHF20 F | TGAGTGGGGACTTCGTGTTC (SEQ ID NO: 38) |
| PHF20 R | GACCAACCGACAGAAGGACT (SEQ ID NO: 39) |
| JAM2 F | TCCACCCCTAGGCTGAAAAG (SEQ ID NO: 40) |
| JAM2 R | GATCGGCTTTGTGTCTGGTC (SEQ ID NO: 41) |

Animals:

All animals were housed at Memorial Sloan Kettering Cancer Center. All animal procedures were completed in accordance with the Guidelines for the Care and Use of Laboratory Animals and were approved by the Institutional Animal Care and Use Committees at Memorial Sloan Kettering Cancer Center.

Generation of Bap1-Deficient and Bap1/Ezh2-Deficient Mice:

Embryonic stem cells targeting exons 6-12 of Bap1 were obtained from the European Conditional Mouse Consortium. A Frt-flanked premature stop cassette containing a lacZ and neomycin cassette was inserted upstream. ES cell clones were expanded and injected into primary blastocysts. Generated mice were crossed to the germline Flp-deleter (The Jackson Laboratory) to excise the Frt-flanked cassette. These mice were subsequently crossed to the IFN-α-inducible Mx1-cre transgenic mice (The Jackson Laboratory) to assess the effects of inducible loss of Bap1 in the hematopoietic system. Bap1 fl/fl, Bap1 fl/+, and Bap1+/+ littermate mice were genotyped by PCR with the primers BAP1-up (actgcagcaatgtggatctg (SEQ ID NO. 1)), BAP1-down (gaaaaggtctgacccagatca (SEQ ID NO. 2)) using the following parameters: 95° C. for 10 min, followed by 40 cycles of 94° C. for 10 s, 65° C. for 40 s, and 72° C. for Imin, and then 72° C. for 5 min. The WT allele was detected at 300 bp while the floxed allele was detected at 500 bp PCR. Excision after IFN-α-induction was confirmed by a PCR with primers to detect the floxed and excised band: BAP1-F (actgcagcaatgtggatctg (SEQ ID NO. 1)), BAP1-F2 (gcgcaacgcaattaatgata (SEQ ID NO. 3)), and BAP1-R (cagtgtccagaatggctcaa (SEQ ID NO. 4)), using the same PCR parameters listed above. Mx1-Cre-Bap1 f/f mice were crossed to Ezh2 f/f mice 12. Mx-cre Bap1f/f conditional and Bap1f/f control mice received four intraperitoneal injections of polyI:polyC of 200 μL of a 1 mg/mL solution. Two weeks after excision, peripheral blood was collected via retroorbital bleeding using heparinized icrohematocrit capillary tubes (Thermo Fisher Scientific). Excision was confirmed and peripheral blood counts were obtained using a HemaVet according to standard manufacturer's instruction. Formalin-fixed paraffin-embedded tissue sections were stained with hematoxylin and eosin (H&E). Deletion of Bap1 was confirmed by genomic excision PCR and Western blot analysis. Tails were submitted to the Transnetyx genotyping service (Cordova, Tenn.) for qPCR-based genotyping for floxed and excised Ezh2 alleles. Excision was confirmed by Western blot.

Xenografts and in vivo EPZ011989 Administration:

Groups of 10 week old NOD-SCID mice were injected subcutaneously in the flank with 6-10×10⁶ mesothelioma cell lines (MSTO-211H, Meso10, H226 and H2452) in a 1:1 mixture of matrigel and media. When tumors reached a size of approximately 60-80 mm³, treatment with either vehicle (0.5% NaCMC+0.1% Tween-80 in water) or EPZ011989 was initiated. Either EPZ011989 or vehicle were given orally BID at a concentration of 500 mg/kg for the duration of the experiment. Tumor volumes were assessed in three dimensions using a caliper. Tumors or lung tissue were extracted following treatment and utilized for Western blotting to assess target inhibition. Pre-established criteria were generated to exclude mice in xenograft experiments if tumors did not form after implantation (75% smaller than the mean of the implanted animals from the same group. Animals were not excluded from drug trials. For all xenograft drug studies, tumor size was followed for 10 days and mice were randomized at this point for tumor size. The genetic Bap1 KO EPZ011989 trial was conducted with randomization utilizing CBC analysis 3 weeks after polyI:polyC and confirming that WBC count averages were equivalent in both vehicle and treated groups. Five animals per group were treated orally with either vehicle (described above) or 500 mg/kg EPZ011989 BID for 16 days. Researchers were not blinded in these experiments.

Histological Analyses:

Mice were sacrificed and autopsied, and then dissected tissue samples were fixed for 24 hours in 4% paraformaldehyde, dehydrated, and embedded in paraffin. Paraffin blocks were sectioned at 4 μm and stained with H&E, Ki67, E-Cadherin, or TUNEL. Images were acquired using an Axio Observer A1 microscope (Carl Zeiss).

Cell Culture:

293T cells were cultured in Dulbecco's modified Eagle's medium (DMEM) supplemented with 10% fetal bovine serum (FBS) and nonessential amino acids. Human leukemia cell lines (SET2) and human mesothelioma cell lines (JMN, Met5a, MSTO-211H, H2373, H226, H2452) were cultured in RPMI-1640 medium supplemented with 10% FBS. MSTO-211H was obtained from ATCC while the remaining mesothelioma lines were generous gifts from Prasad Adusumilli.

RNA Isolation, SMARTer Amplification, Proton Transcriptome Sequencing and Analysis:

Bone marrow cells were FACS sorted for GMPs (Lin⁻c⁻Kit⁺ Sca1⁻CD34⁻Fcγ⁺) using the FACS Aria. Total RNA from 200-500K cells was extracted using TRIzol RNA Isolation Reagents (cat#15596-026, Life Technologies). Quality of RNA was ensured before amplification by analyzing 20-50 pg of each sample using the RNA 6000 pico kit and a bioAnalyzer (Agilent). 10 ng of high quality (RIN>8) total RNA was subsequently amplified using the SMARTER® Universal Low Input RNA Kit for Sequencing (Clonetech Laboratory, cat#634940) according to instructions provided by the manufacturer. Amplified material underwent whole transcriptome Library preparation according to the Ion Total RNA-Seq Kit v2 protocol (Life Technologies), with 16 cycles of PCR. Samples were barcoded and template-positive ION PI™ ION SPHERE™ Particles (ISPs) were prepared using the ion one touch system II and ION PI™ Template OT2 200kit v2 Kit (Life Technologies). Enriched particles were sequenced on a Proton sequencing system using 200 bp version 2 chemistry. An average of 70 to 80 million reads per sample were generated and 76 to 82% of the reads mapped to mRNA bases. RAW output BAMs were converted back to FASTQ using PICARD Sam2Fastq. Then the reads are first mapped to the mouse genome using maStar. The genome used was MM9 with junctions from ENSEMBL (Mus_musculus.NCBIM37.67) and a read overhang of 49. Then any unmapped reads were mapped to MM9 using BWA MEM (version 0.7.5a). The two mapped BAMs were then merged and sorted and gene level counts were computed using htseq-count (options -s y -m intersection-strict) and the same gene models (Mus_musculus.NCBIM37.67). Raw data was uploaded to the GEO database with the following accession number GSE61360.

Histone Extraction, Histone ELISAs, Histone Western Blots, and Histone LC/MS:

Histones were extracted by standard extraction techniques or overnight using the Active Motif Histone Extraction Minikit (40026). Histone ELISAs were conducted using the trimethyl K27 Elisa Kit (Active Motif, 53106) normalized to a H3K27me3 standard curve and total H3 protein. Histone Western blots were conducted with 3-5 μg of histones. For Histone LC/MS, 12 million control and Bap1 KO cells were lysed, nuclei were isolated and histones were extracted using 0.4N H2SO4 and chemically derivatized using propionic anhydride, as previously described 26. Histones were then digested with trypsin and separated by nano-liquid chromatography (75 μm i.d., 15 cm long, packed with MagicC18aq media, dp 3μ) coupled to a TSQ Quantum Ultra mass spectrometer. Data were analyzed with Skyline 27 and relative quantification was performed by peak area.

Chromatin Preparation and Immunoprecipitation, ChIP Library Preparation and Sequencing, and Analysis of ChIP-Seq Data:

Bone marrow cells were enriched for c-Kit+ cells using the EasySep Mouse Hematopoietic cell Enrichment Kit (Stem Cell Technologies, 19756). 5×10⁶ cells were fixed in a 1% methanol-free formaldehyde solution and then resuspended in SDS lysis buffer. Lysates were sonicated in an E220 focused-ultrasonicator (Covaris) to a desired fragment size distribution of 100-500 base pairs. IP reactions were performed using anti-trimethyl H3K27 (Cell Signaling, 9733), antimonomethyl H4K20 (Abeam, 9051), and IgG (Santa Cruz, 2027) each on approximately 400,000 cells as previously described (Krivtsov et al., 2008). ChIP assays were processed on an SX-8G IP-STAR Compact Automated System (Diagenode) using a Direct ChIP protocol as described elsewhere (O'Geen et al., 2011). Eluted chromatin fragments were then de-crosslinked and the DNA fragments purified using Agencourt AMPure XP beads (Beckman Coulter).

Barcoded libraries were prepared from the ChIP-enriched and input DNA using a NEBNext ChIP-Seq Library Prep Master Mix Set for Illumina (New England Biolabs) and TruSeq Adaptors (Illumina) according to manufacturer's instructions on an SX-8G IP-STAR Compact Automated System (Diagenode). Phusion High-Fidelity DNA Polymerase (New England Biolabs) and TruSeq PCR Primers (Illumina) were used to amplify the libraries, which were then purified to remove adaptor dimers using AMPure XP beads and multiplexed on the HiSeq 2000 (Illumina).

Reads were quality and adapter-trimmed using ‘trim_galore’ before aligning to mouse assembly mm9 with bowtie2 using the default parameters. Aligned reads with the same start position and orientation were collapsed to a single read before subsequent analysis. Density profiles were created by extending each read to the average library fragment size and then computing density using the BEDTools suite. Enriched regions were discovered using MACS 1.4 with default parameters, and scored against matched input libraries. All genome browser tracks and read density tables were normalized to sequencing depth. For comparison of ChIP-seq samples in control and KO conditions, the signals of three replicates per condition were tested using either the Mann-Whitney U test or the t-test. Cluster analysis was performed on normalized count data in Matlab with the kmeans clustering package. Motif analysis was performed in Homer using default parameters for the findMotifsGenome program.

Western Blot and Immunoprecipitation:

Cells were lysed for Western blot and immunoprecipitation experiments in the following buffer: 150 mM NaCl, 20 mM Tris (pH 7.4), 5 mM EDTA, 1% Triton, protease arrest (EMD) and phosphatase inhibitors (Calbiochem). To perform immunoprecipitations in the presence of benzonase, cells were lysed in the BC-300 buffer: 20 mM Tris (pH 7.4), 10% glycerol, 300 mM KCl, 0.1% NP-40. The cleared lysate was treated with MgCl2 to 2.5 mM and benzonase was added at 1250 U/mL. The lysate was incubated for 1 hour with rotation and the reaction was terminated by adding 5 mM EDTA. DNA digestion was confirmed by running lysate on an ethidium bromide gel before setting up the immunoprecipitation experiment. Antibodies used included: BAP1 (C-4; Santa Cruz sc-28383), EZH2 (Active Motif, 39933, Active Motif, 39901, or Millipore, 07-689), SUZ12 (Abeam, Ab12073), ASXL1 (N-13; Santa Cruz sc-85283), L3MBTL2 (Active Motif, 39569), Myc-Tag (Cell Signaling, 2276), Tubulin (Sigma, T9026), H3K27me3 (Abcam, 6002 or Millipore, 07-449), H3 (Abcam, Ab1791), and H4K20me1 (Abcam, Ab9051).

Flow Cytometry Analyses and Antibodies:

Surface marker staining of live bone marrow and spleen cells was conducted by first lysing cells with ammonium chloride-potassium bicarbonate lysis buffer and washing cells with phosphate buffered saline (PBS). Cells were stained with antibodies in PBS for 20 minutes on ice. For hematopoietic stem and progenitor staining, cells were stained with a lineage cocktail including CD4 (RM4-5), CD3 (17A2), B220 (RA3-6B2), NK1.1 (PK136), Gr-1 (RB6-8C5), Cd11b (M1/70), and Ter119, allowing for mature lineage exclusion from the analysis. Cells were also stained with antibodies against c-Kit (2B8), Sca-1 (D7), FcγRII/III (2.4G2), and CD34 (RAM34). To assess the composition of the mature mononuclear cells, Mac1, Gr-1, B220, and CD4/CD3 were used. Cell cycle analysis was conducted by staining cells with the hematopoietic stem and progenitor mix described above. Cells were fixed using the FIX and PERM kit (Invitrogen cat#GAS-003). Cells were stained with Ki67 after fixation and then stained with DAPI before analysis on the LSR Fortessa.

Plasmids:

The cDNA full-length clone of human FLAG-L3MBTL2 was obtained from Addgene (Plasmid 28232). The Myc-His tagged ubiquitin construct was a generous gift from Xuejun Jiang. The cDNA human full-length clone of HA-FLAG BAP1 was obtained from Addgene (Plasmid 22539). The 3× FLAG-tagged BAP1 construct was a generous gift from Marc Ladanyi. Deubiquitinase mutant constructs (C91A, C91S) were generated using Agilent site-directed mutagenesis kits and confirmed by full-length DNA sequencing. Short-hairpin RNAs were obtained from the RNAi Consortium (TRC) in a pLKO.1 puromycin vector. Sequences for the short-hairpins were as follows: human BAP1 (TRC Oligo IDs: TRCN0000078702 and TRCN0000078698), mouse BAP1 (TRCN0000030719 and TRCN0000030720), human L3MBTL2 (TRCN0000021724 and TRCN0000021726) and a control pLKO.1-puromycin vector encoding an shRNA for luciferase (shLUC).

Ubiquitin Assays:

HEK293T cells were seeded in a 10-cm dish and 24 hours later were transduced with 4 μg of a Myc-His-Ubi expression construct and control, 1 μg L3MBTL2 and/or 1-10 μg BAP1-GFP overexpression constructs. Forty-eight hours after the transfection, cells were lysed in a Guanidine HCl based lysis buffer 6 M guanidine, 0.1 M NaH2PO4, 10 mM Tris, pH 8.0, and 10 mM BME. His-Ubi proteins were purified by incubation by 20 μL of Ni-NTA agarose (Qiagen) for 4 hours at room temperature. Beads were washed sequentially with 1 mL of 4 wash buffers: buffer A 6 M guanidine, 0.1 M NaH2PO4, 10 mM Tris, pH 8.0, 10 mM BME, and 0.2% Triton-X, buffer B 8 M urea, 0.1 M NaH2PO4, 10 mM Tris, pH 8.0, 10 mM BME, and 0.2% Triton-X, buffer C 0.1 M NaH2PO4, 10 mM Tris, pH 6.3, 10 mM BME, and 0.2% Triton-X, and buffer D 0.1 M NaH2PO4, 10 mM Tris, pH 6.3, 10 mM BME, and 0.1% Triton-X. All buffers were supplemented with 15 mM imidazole. His-tagged proteins were purified from the beads by boiling with 2×SDS Laemmli buffer supplemented with imidazole. Proteins were then analyzed by Western blot.

In Vitro Colony Forming Assays:

Cells were sorted for Lin⁻c-Kit⁺Sca1⁺ cells using the FACSAria. 100 cells were plated in duplicate in methylcellulose (MethoCult GF M3434, Stem Cell Technologies). Colonies were counted 14 days after plating and colonies were collected by washing with PBS. Cells were then lysed for RNA and histone extraction.

Transient Transfection:

293T cells were transfected with indicated constructs with X-treme gene transfection reagent (Roche). Protein and/or histones were extracted 48-72 hours after transfection.

Invasion Assays:

Mesothelioma cells (MSTO-211H, H2373, H226 and H2452) were seeded in T75 flasks (100,000 cells). 12 hours later the plated cells were treated with GSK126 (0-2 μM) (Chemitek) and then left to proliferate for 7 days. 250,000 treated cells were then placed on the top of a Matrigel invasion chamber (BD Biosciences, cat no. 354480) in serum free media while the lower chamber contained media with serum. 22 hours later the cells on the bottom of the membrane were stained with crystal violet and quantitated with ImageJ.

Luciferase Assays:

293T cells were transiently transfected with the pGL3 EZH2 promoter reporter construct (generous gift from Naomi Goldfinger) and a Switchgear Renilla control construct in addition to EV, BAP1, and L3MBTL2 constructs. Cells were assessed for luciferase activity using the DualLuciferase Reporter Assay System (Promega). Cells were seeded in 24 well plates and were cotransfected with 200 ng pGL3-EZH2-Luciferase, 200 ng of the Renilla luciferase control construct, and 500 ng of experimental constructs. Cells were incubated 48 hours after the transfection, lysed for 15 minutes at room temperature and luciferase activity was assessed on a luminometer. The Firefly luciferase readings were normalized to the Renilla transfection control.

Statistical Analyses:

The Student t-test with Welch's correction was used to analyze statistical significance unless described in the text. Prizm GraphPad Software was used for statistical calculations. Error was calculated using SEM, *p<0.05, **p<0.005.

### 7.2 Results

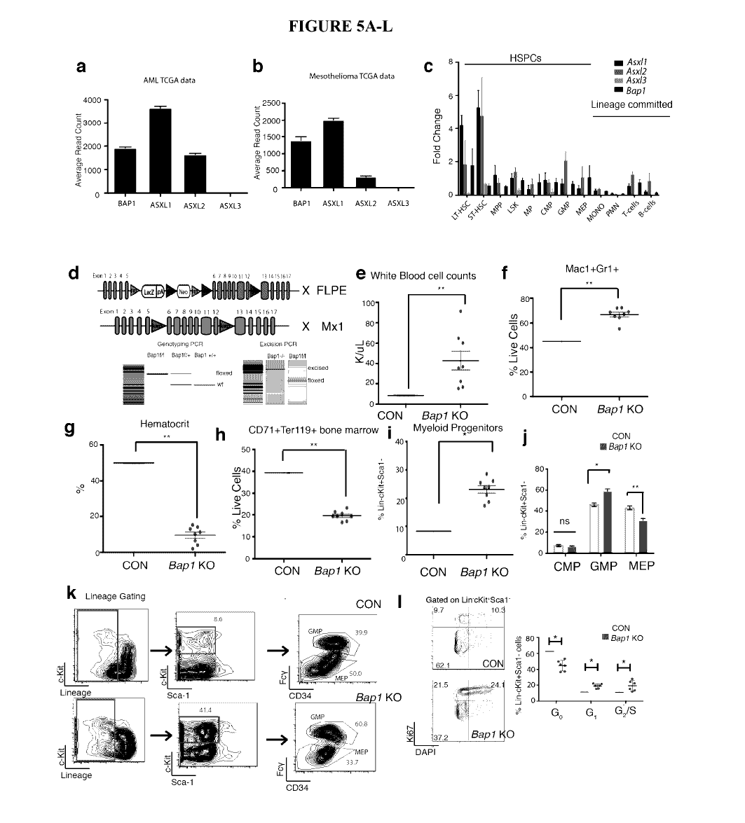

Genomic studies identified somatic mutations in the tumor suppressors ASXL1 and BAP1 in different malignancies. The Drosophila ASXL1 homolog Asx and the BAP1 homolog Calypso form a complex which removes H2AK119Ub (Scheuermann et al., 2010). However, the BAP1-ASXL1 complex has not been shown to have a role in BAP1-mutant transformation. Inactivating mutations in ASXL1 are most common in myeloid malignancies (Abdel-Wahab et al., 2011; Bejar et al., 2011; Gelsi-Boyer et al., 2009), whereas recurrent BAP1 mutations are commonly observed in mesothelioma (Bott et al., 2011), renal cell carcinoma (Pena-Llopis et al., 2012), and metastatic uveal melanoma (Harbour et al., 2010) suggesting BAP1 and ASXL1 have distinct roles in tumor suppression. These mutational profiles cannot be explained by differential tissue-specific BAP1 and ASXL1 expression (FIG. 5A-C). This Example identifies the mechanisms by which BAP1 loss leads to transformation, independent of ASXL1, and identifies the therapeutic vulnerabilities in BAP1-mutant cancer cells.

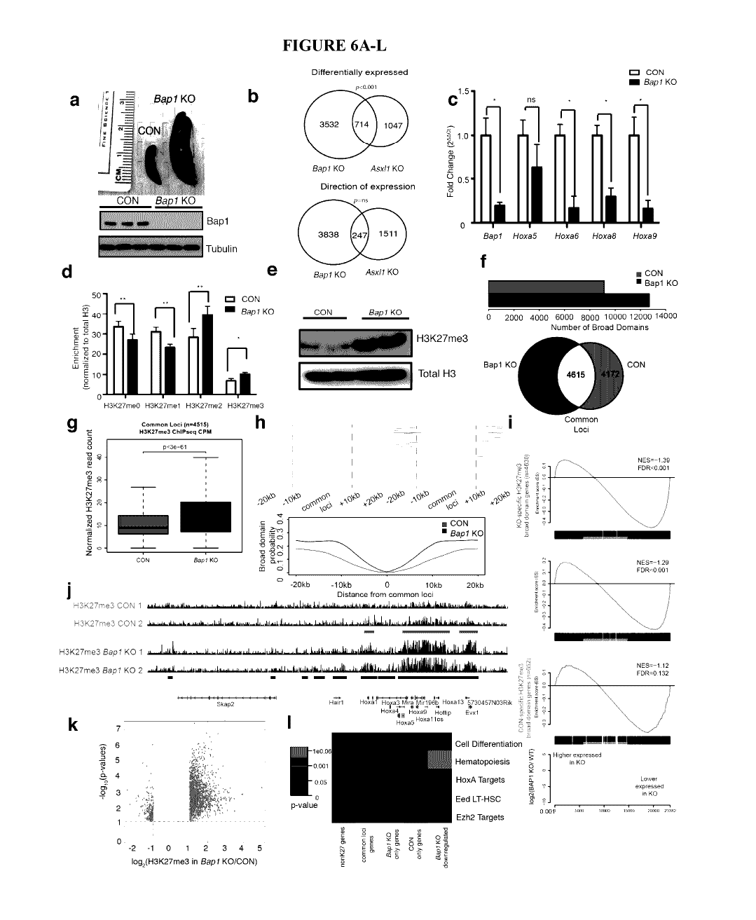

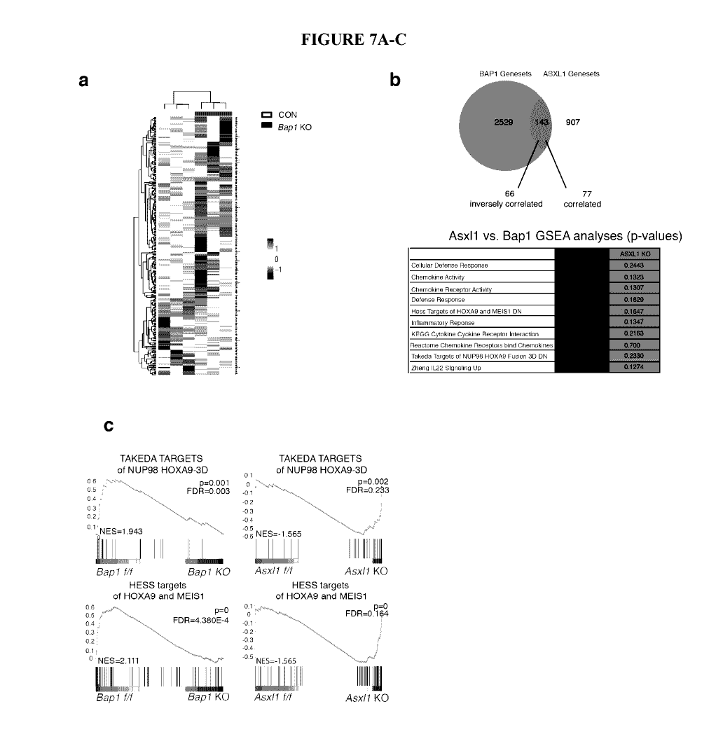

Recent studies have shown that somatic loss of Bap1 can promote hematopoictic transformation (Dey et al., 2012). The impact of conditional Bap1 deletion on gene expression and chromatin state in hematopoietic cells was investigated (FIG. 5A, B). Conditional deletion of Bap1 was generated using the scheme shown in FIG. 5D. MX-Cre, a recombinase that drives Bap1 deletion in hematopoietic tissues following induction in the adult animal was used. Bap1 loss led to a fully penetrant myeloproliferative disease with splenomegaly (FIG. 6A), leukocytosis (FIGS. 5E,F), anemia (FIGS. 5G,H) and granulocyte macrophage progenitors (GMPs) expansion (FIGS. 5I-K). For example, in Bap1 knockout (KO) mice, the size, e.g., weight and length, of the spleen was observed to be larger in size than BAP1 wild-type mice (FIGS. 6A, 10C and 11C). An increase in proliferation and cell cycle progression of Bap1-deficient myeloid progenitors was also observed (FIGURE SL). RNA sequencing analysis revealed the majority of differentially expressed genes in Bap1-deficient GMPs had reduced expression (p-adj<0.001) (FIG. 7A). Although significant overlap between the set of differentially expressed genes in Bap1 and Asxl1 KO progenitors was observed, in many cases a paradoxical inverse effect on gene expression was observed (FIG. 6B). Gene set enrichment analysis (GSEA) identified inversely impacted gene sets enriched in Bap1 and Asxl1 KO progenitors (Abdel-Wahab et al., 2013) (FIG. 7B). ASXL1 silencing leads to increased expression of HoxA cluster genes consistent with reduced PRC2 activity (Abdel-Wahab et al., 2012). By contrast, reduced expression of HoxA gene members (FIG. 6C) and decreased expression of HoxA gene signatures in Bap1-deficient cells (FIG. 7C) were observed. These data demonstrate that loss of Asxl1 and Bap1 have opposite effects on gene regulation.

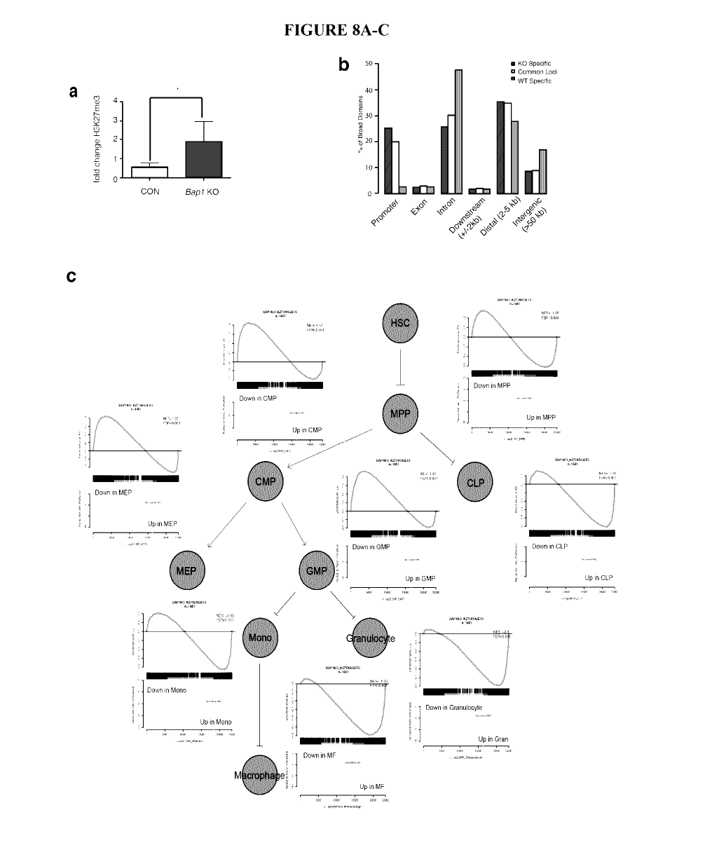

ASXL1 directly interacts with the PRC2 complex and ASXL1 depletion reduces global and site-specific H3K27me3 (Abdel-Wahab et al., 2012). Given the divergent effects of Asxl1 and Bap1 loss on gene expression, the impact of Bap1 deletion on H3K27me3 was investigated. H3K27me3 levels were increased in Bap1 KO cells by histone mass spectrometry (FIG. 6D), Western blot (FIG. 6E), and ELISA (FIG. 8A). H3K27me3 chromatin immunoprecipitation sequencing (ChIP-Seq) revealed a global increase in Bap1 KO mice (FIG. 6F), with an increased number of H3K27me3 broad domains (Beguelin et al., 2013) (FIG. 6G), and increased H3K27me3 broad domain “spreading” into nearby loci (FIG. 6H). This H3K27me3 increase and spreading is well illustrated within the HoxA locus in Bap1 KO cells (FIG. 6I). The sites marked with H3K27me3 in Bap1 KO cells, primarily occurred in gene promoter regions (FIG. 8B) and genes with H3K27me3-occupied promoters were enriched for enhanced repression (FDR<0.001) (FIG. 63). Similar findings in purified GMPs were observed (FIG. 6K). Genes dysregulated by Bap1 KO-associated H3K27me3 and gone repression were implicated in EZH2-dependent regulation, lineage commitment/differentiation and proliferation (FIG. 6L, FIG. 8C). BAP1 silencing increased H3K27me3 (FIG. 9A), and re-expression of BAP1 in Bap1-deficient cells reduced H3K27me3 levels (FIG. 9B). By contrast, a deubiquitinase-deficient BAP1 allele did not reduce H3K27me3 (FIG. 9C), demonstrating alterations in H3K27me3 are due to BAP1 catalytic activity.

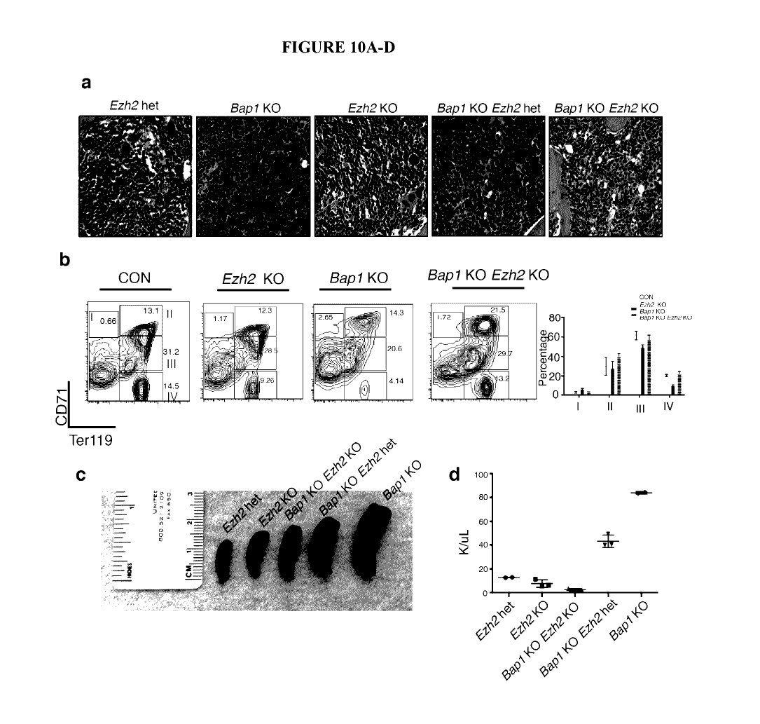

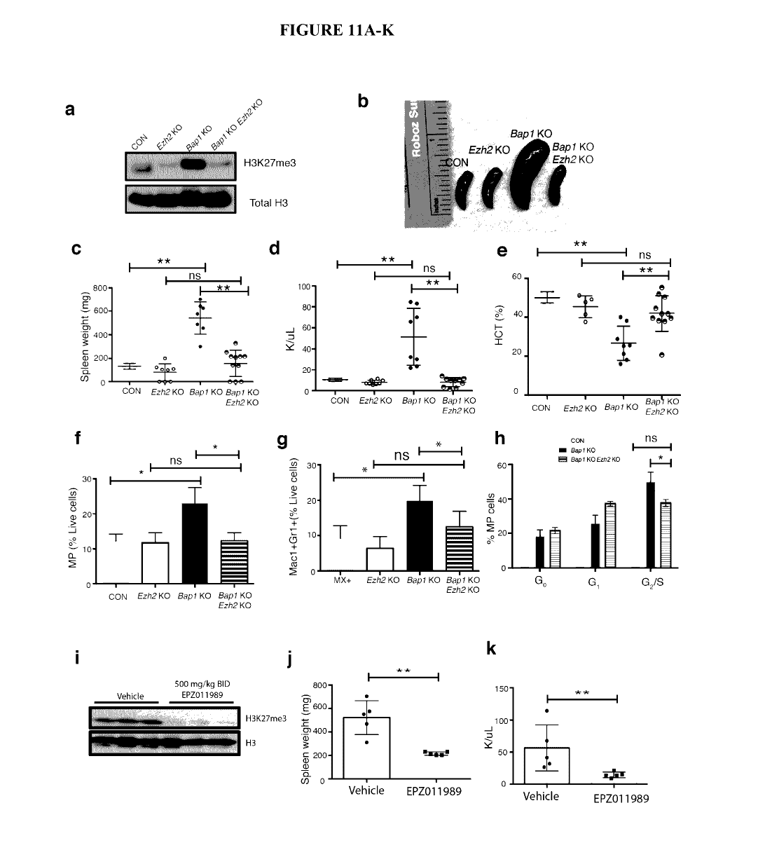

Next, the role of PRC2-mediated H3K27me3 on BAP1-dependent transformation was assessed by investigating the impact of Ezh2 loss (Su et al., 2003) on transformation in vivo. Ezh2 deletion reduced H3K27me3 levels in Bap1/Ezh2-deficient mice compared to Bap1-knockout mice (FIG. 11A). Ezh2 deletion abrogated the myeloid malignancy induced by Bap1 loss (FIG. 10A), with reduced splenomegaly (FIGS. 11B,C), leukocytosis (FIG. 11D) and anemia (FIG. 11E). Concomitant Bap1/Ezh2 loss reduced myeloid progenitor expansion (FIG. 11F), reduced the proportion of Mac1⁺Gr1⁺ myeloid cells (FIG. 11G) and restored erythroid differentiation (CD71⁺Ter119⁺) (FIG. 10B). Decreased proliferation of Bap1/Ezh2 deficient progenitors was observed (FIG. 11H). Ezh2 haploinsufficiency reduced, but did not abrogate, Bap1-deficient myeloproliferation (FIGS. 10C, 10D) consistent with a dose-dependent requirement for Ezh2. Consistent with the genetic data, treatment of Bap1 KO mice with the small molecule inhibitor EPZ011989 (Campbell et al., 2015) decreased H3K27me3, splenomegaly, and white blood cell counts (FIGS. 11I-K). These data demonstrate that PRC2, and specifically Ezh2 activity, is required for Bap1-deficient myeloid transformation.

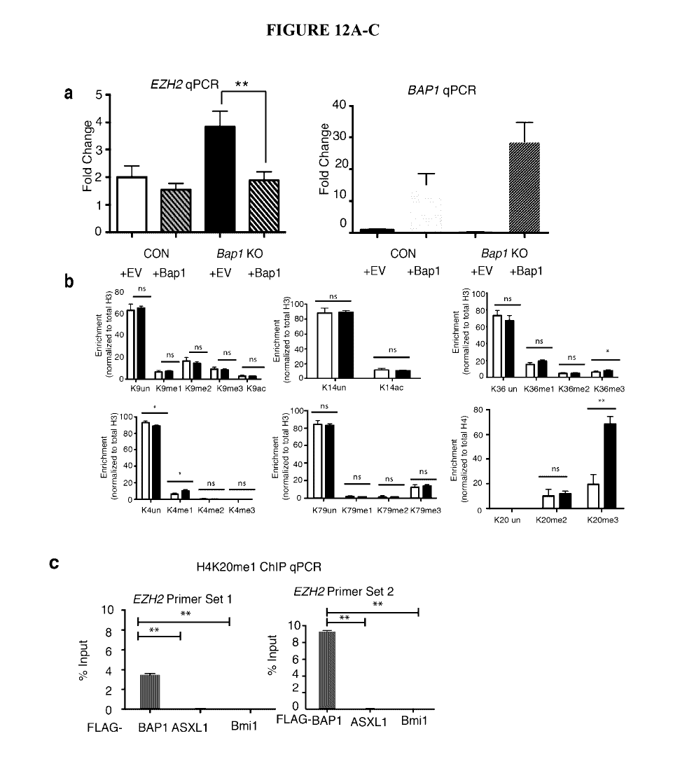

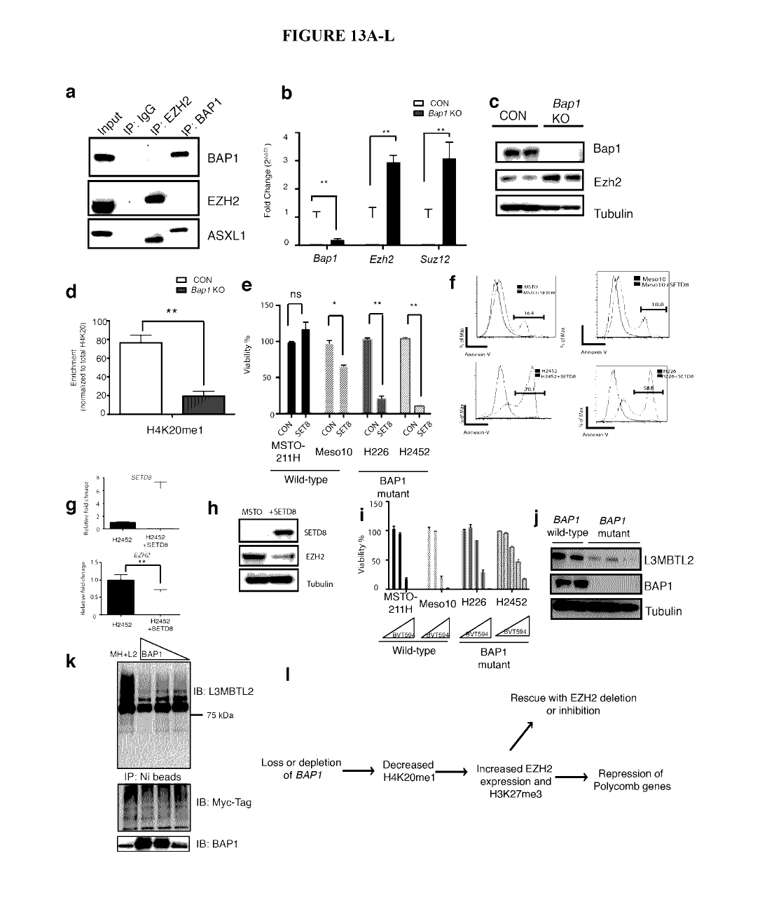

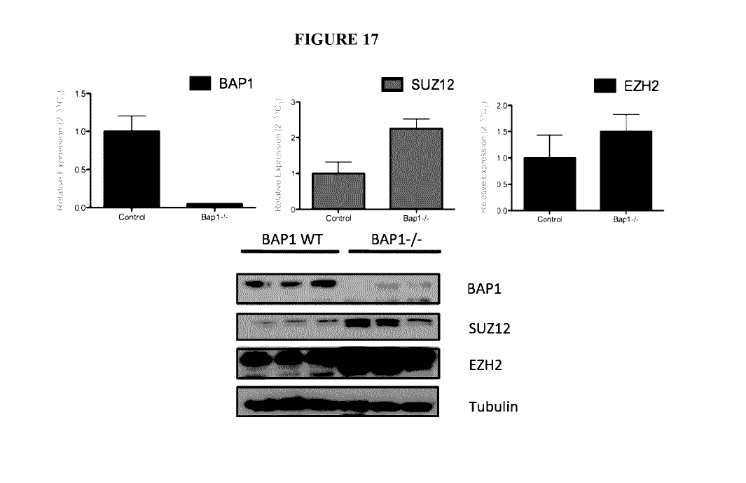

Next, the mechanism by which Bap1 deletion increased H3K27me3 levels was investigated. In contrast to the reported interactions between ASXL1 and BAP1 and between ASXL1 and PRC2, an interaction between BAP1 and EZH2 by co-IP was not identified (FIG. 13A). An increase in mRNA and protein expression of Ezh2 and Suz12 was observed (FIGS. 13B,C) consistent with a role for Bap1 in regulating PRC2 expression. In addition, analysis of sorted Lin⁻Sca⁺Kit⁺ cells (a population containing the hematopoietic stem cells) from the whole bone marrow of Bap1 KO mice showed significant increases in Suz12 and Ezh2 RNA expression compared to wild-type BAP1 mice (FIG. 17). Further, whole bone marrow western blots revealed an increase in the protein expression of EZH2 and SUZ12 in Bap1 KO cells compared to control cells (FIG. 17). Re-expression of BAP1 in Bap1 KO bone marrow cells reduced Ezh2 mRNA expression to normal levels (FIG. 12A). It was hypothesized that Bap1 loss might directly alter other histone marks, which would then alter chromatin state at key target loci, including EZH2. Histone mass spectrometry revealed a marked decrease in H4K20me1 in Bap1 KO cells (FIG. 13D) compared to other measured histone marks (FIG. 12B). Expression of BAP1, but not ASXL1 or BM11, increased H4K20me1 at the EZH2 locus (FIG. 12C). It was therefore hypothesized that loss of the H4K20me1 mark may have an important role in BAP1-dependent gene expression. SETD8 is the only known methyltransferase that places H4K20me1 (Nishioka et al., 2002). Expression of SETD8 in BAP1-mutant mesothelioma cells (H226, H2452) increased apoptosis and reduced proliteration, whereas wild-type (MSTO-211H and Meso10) cells were unaffected (FIGS. 13E,F). SETD8 overexpression in mesothelioma cells decreased EZH2 mRNA and protein expression (FIGS. 13G,H). BAP1 wild-type cell lines were more sensitive to a SETD8 inhibitor (Blum et al., 2014) (BVT594) than BAP1-mutant cell lines (FIG. 13I).

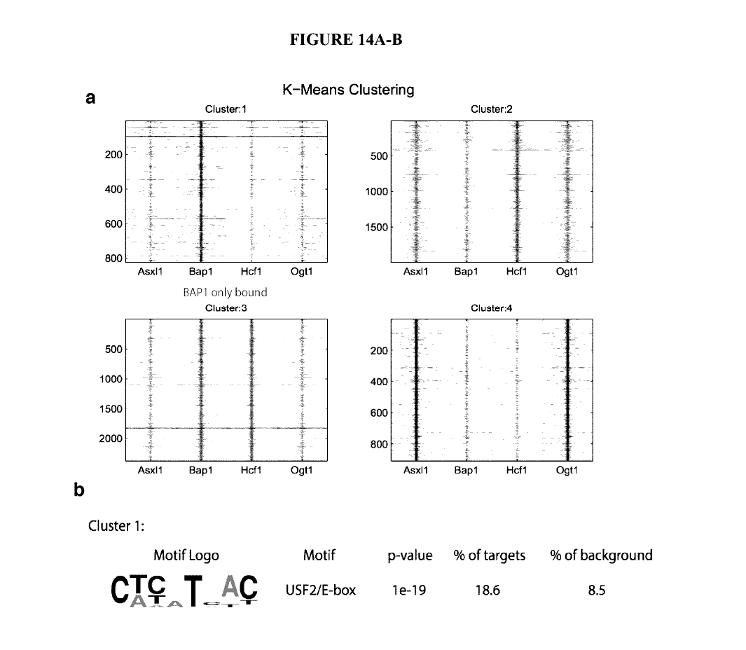

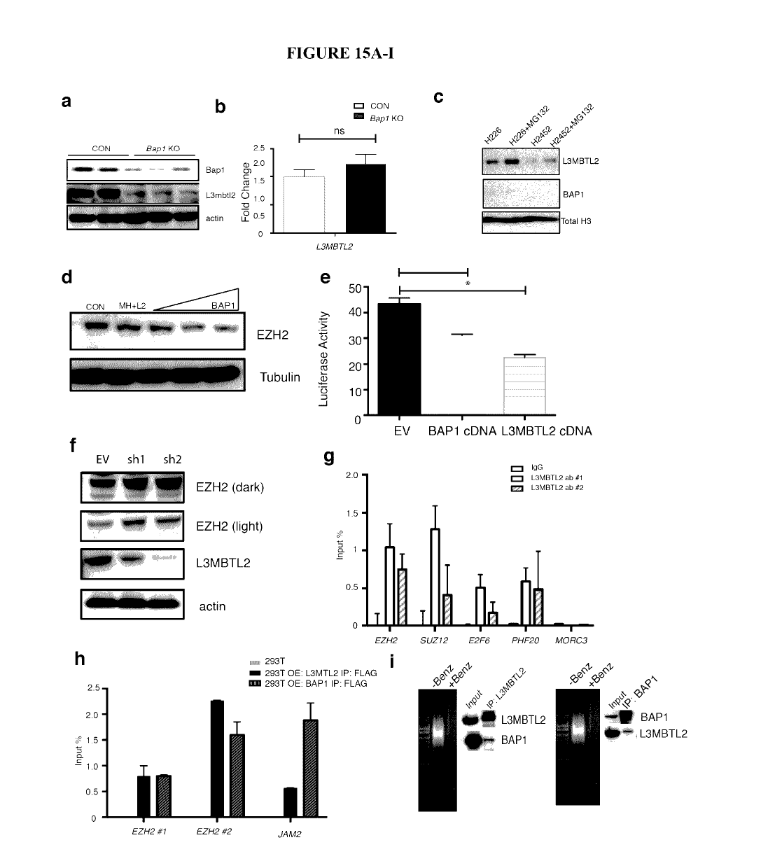

It was hypothesized that BAP1 deubiquitinates a chromatin modulator that regulates H4K20me1. Analysis of ChIP-Seq data (Abdel-Wahab et al., 2013; Dey et al., 2012) identified a cluster of genes with Bap1 occupancy, but not Asxl1 binding (Cluster 1) and were enriched for an E-box motif (FIGS. 14A,B). Previous studies have shown the atypical polycomb proteins L3MBTL1 and L3MBTL2 bind E-box motifs, and can bind and maintain H4K20me1 (Guo et al., 2009; Qin et al., 2012; Trojer et al., 2011; Trojer et al., 2007). L3mbtl1-deficient mice have no overt phenotype (Qin et al., 2010), whereas L3mbtl2-deficient mice are embryonic lethal similar in timing to Bap1 loss (Dey et al., 2012; Qin et al., 2012). Therefore, whether Bap1 loss led to alterations in L3mbtl2 expression was investigated. L3mbtl2 protein but not RNA expression was reduced in Bap1 KO hematopoietic cells (FIGS. 15A,B) and in BAP1-mutant mesothelioma cells compared to BAP1 wild-type mesothelioma cells (FIG. 13J). L3MBTL2 ubiquitination was reduced in cells expressing BAP1 (FIG. 13K) and proteasome inhibitor treatment increased L3MBTL2 stability in BAP1-mutant cells (FIG. 15C). L3MBTL2 expression decreased EZH2 protein levels with and without BAP1 co-expression (FIG. 15D) and expression of BAP1 or L3MBTL2 overexpression reduced EZH2 promoter activity (FIG. 15E). Conversely, L3MBTL2 silencing increased expression of EZH2 (FIG. 15F). An enrichment for L3MBTL2 and BAP1 at the EZH2 locus was observed in cells expressing L3MBTL2 and BAP1 (FIGS. 15G,H). Without being bound to a particular theory, these data suggest that BAP1 and L3MBTL2 interact (FIG. 15I) and co-occupy the EZH2 locus. BAP1 loss leads to reduced L3MBTL2 stability and increased EZH2 transcriptional output (FIG. 13L).

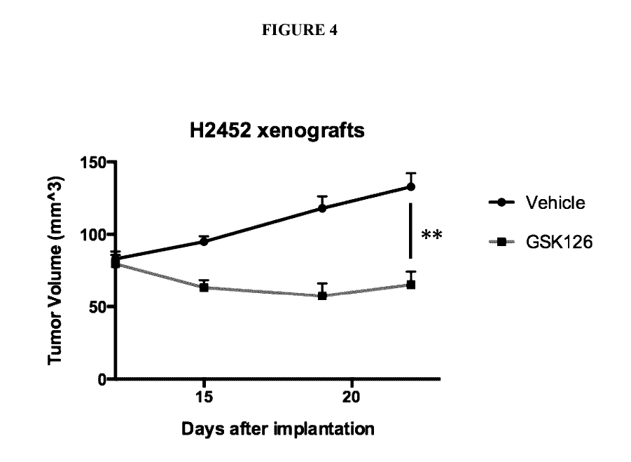

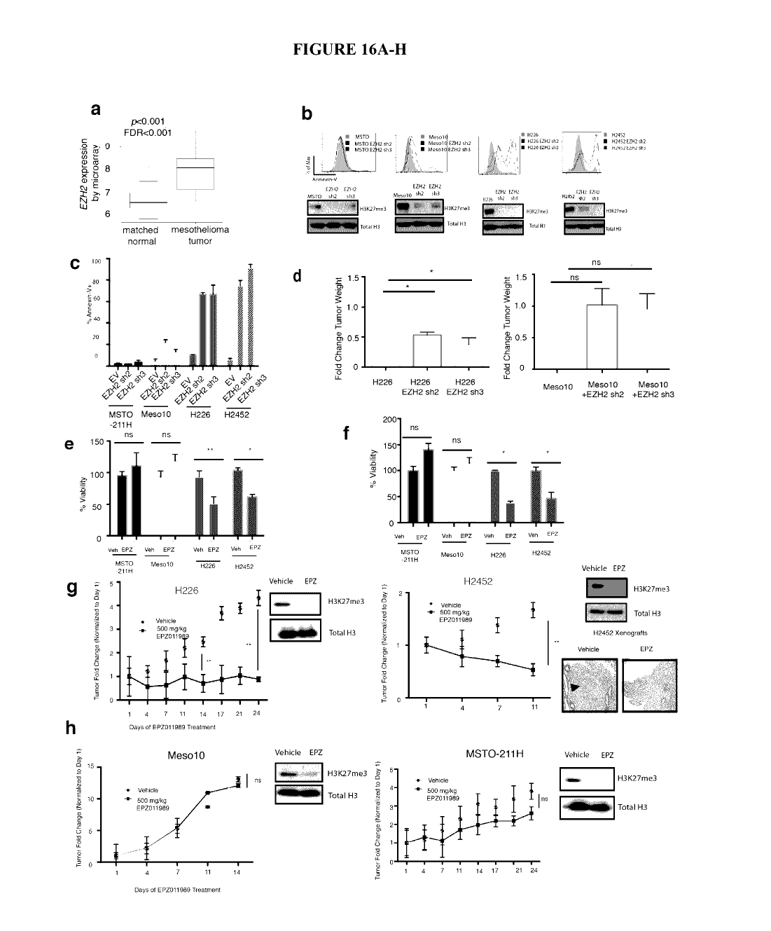

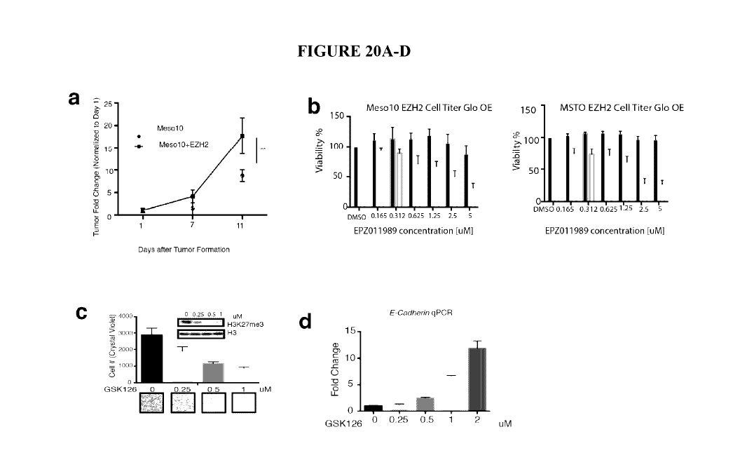

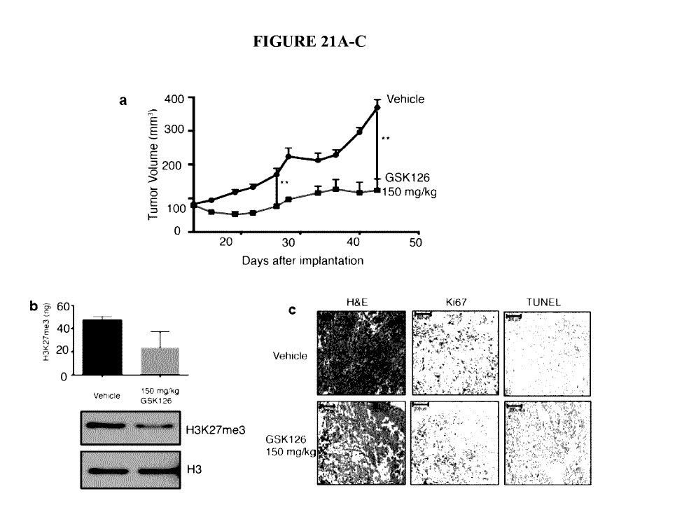

Analysis of TCGA data revealed that EZH2 mRNA expression was increased in mesothelioma (FIG. 16A). Next, whether EZH2 inhibition might inhibit the survival of BAP1-mutant mesothelioma cell lines was assessed. EZH2 silencing induced apoptosis in BAP1-mutant cell lines, whereas wild-type cell lines continued to proliferate (FIGS. 16B,C). EZH2 silencing abrogated in vivo tumor formation of BAP1-mutant but not wild-type cell lines (FIG. 16D). Overexpression of EZH2 in BAP1 wild-type cell lines increased proliferation (FIG. 20A) and sensitivity to EZH2 inhibition (FIG. 20B). BAP1-mutant cell lines were more sensitive to EZH2 inhibition (EPZ011989) in vitro both in 2D (FIG. 16E) and 3D culture (FIG. 16F). Next, the impact of EZH2 inhibition in vivo was assessed. EZH2 inhibition significantly reduced BAP1-mutant tumor size compared to vehicle treated mice (FIG. 16G), whereas wild-type tumors were less/not responsive to EZH2 inhibition (FIG. 16H), despite similar effects on H3K27me3. EZH2 inhibition abrogated pulmonary metastasis in a BAP1-mutant mesothelioma cell line with metastatic potential (FIG. 16G) consistent with a role for BAP1/EZH2 in metastasis (Harbour et al., 2010). EZH2 inhibition reduced invasion and increased E-Cadherin expression in vitro (FIGS. 20C-D). Next, the impact of EZH2 inhibition using the BAP1 inhibitor GSK126 was assessed. BAP1-mutant mesothelioma cells were injected into the flank of NOD-SCID mice, and then initiated treatment with either vehicle or 150 mg/kg GSK126 after tumor formation. EZH2 inhibition significantly reduced tumor size compared to vehicle treated mice (FIG. 21A and FIG. 4). GSK126 treatment significantly attenuated H3K27me3 in BAP1-mutant cells in vivo (FIG. 21B). Pathologic analysis revealed that EZH2 inhibition was associated with reduced Ki67 staining and increased TUNEL staining (FIG. 21C). These data indicate that EZH2 represents a potential therapeutic target in BAP1-mutant cancer cells.

The identification of oncogenic EZH2 mutations (Morin at al., 2010; Morin et al., 2011; Pasqualucci at al., 2011) has led to the development of mutant-specific epigenetic therapies. However, most mutations in epigenetic regulators result in loss-of-function, such that they do not represent tractable direct therapeutic targets. EZH2 inhibitors have recently entered clinical trials (McCabe et al., 2012) and the disclosed data suggest that BAP1 loss results in a mutation-specific dependency in PRC2 that should be further explored in preclinical and clinical studies. These data resonate with recent studies suggesting a role for PRC2 inhibition in SWI/SNF-mutant rhabdoid tumors (Alimova at al., 2013; Knutson et al., 2013) and analyses showing BAP1 mutations are mutually exclusive with SWI/SNF mutations (Wilson at al., 2010). These data suggest that detailed studies of mutations in epigenetic regulators can be used to inform the development of therapies that reverse mutant-specific effects on epigenetic state in different malignant contexts.

### 7.3 Discussion

BAP1 and ASXL1 interact to form a polycomb deubiquitinase complex that can remove monoubiquitin from historic H2A lysine 119 (H2AK119Ub). However, BAP1 and ASXL1 are mutated in distinct cancer types, consistent with independent roles in regulating epigenetic state and in malignant transformation. In this Example, it is demonstrated that Bap1 loss results in increased trimethylated histone H3 lysine 27 (H3K27me3), elevated Ezh2 expression, and enhanced repression of Polycomb Repressive Complex 2 (PRC2) targets. These findings are in contrast to the reduction in H3K27me3 seen with Asxl1 loss. Conditional deletion of Bap1 and Ezh2 in vivo abrogates the myeloid progenitor expansion induced by Bap1 loss alone. Loss of Bap1 results in a marked decrease in H4K20 monomethylation (H4K20me1). Consistent with a role for H4K20me1 in EZH2 transcriptional regulation, expression of SETD8, the H4K20me1 methyltransferase, reduces EZH2 expression and abrogates the proliferation of BAP1-mutant cells. Further, mesothelioma cells that lack BAP1 are sensitive to EZH2 pharmacologic inhibition, suggesting a novel therapeutic approach for BAP1-mutant malignancies.

### 8. REFERENCES

Abdel-Wahab, O., Adli, M., LaFave, L. M., Gao, J., Hricik, T., Shih, A. H., Pandey, S., Patel, J. P., Chung, Y. R., Koche, R., et al. (2012). ASXL1 mutations promote myeloid transformation through loss of PRC2-mediated gene repression. Cancer Cell 22, 180-193.
Abdel-Wahab, O., Gao, J., Adli, M., Dey, A., Trimarchi, T., Chung. Y. R., Kuscu, C., Hricik, T., Ndiaye-Lobry, D., Lafave, L. M., et al. (2013). Deletion of Asxl1 results in myelodysplasia and severe developmental defects in vivo. J Exp Med 210, 2641-2659.
Abdel-Wahab, O., Pardanani, A., Patel, J., Wadleigh, M., Lasho, T., Heguy, A., Beran, M., Gilliland, D. G., Levine, R. L., and Tefferi, A. (2011). Concomitant analysis of EZH2 and ASXL1 mutations in myelofibrosis, chronic myelomonocytic leukemia and blast-phase myeloproliferative neoplasms. Leukemia 25, 1200-1202.
Alimova, I., Birks, D. K., Harris, P. S., Knipstein, J. A., Venkataraman, S., Marquez, V. E., Foreman, N. K., and Vibhakar, R. (2013). Inhibition of EZH2 suppresses self-renewal and induces radiation sensitivity in atypical rhabdoid teratoid tumor cells. Neuro Oncol 15, 149-160.
Beguelin, W., Popovic, R., Teaster, M., Jiang, Y., Bunting, K. L., Rosen, M., Shen, H., Yang, S. N., Wang, L., Ezponda, T., et al. (2013). EZH2 is required for germinal center formation and somatic EZH2 mutations promote lymphoid transformation. Cancer Cell 23, 677-692.
Bejar, R., Stevenson, K., Abdel-Wahab, O., Galili, N., Nilsson, B., Garcia-Manero, G., Kantajian, H., Raza, A., Levine, R. L, Neuberg, D., et al. (2011). Clinical effect of point mutations in myelodysplastic syndromes. The New England journal of medicine 364, 2496-2506.
Blum, G., Ibanez, G., Rao, X., Shum, D., Radu, C., Djaballah, H., Rice, J. C., and Luo, M. (2014). Small-molecule inhibitors of SETD8 with cellular activity. ACS Chem Biol 9, 2471-2478.
Bott, M., Brevet, M., Taylor, B. S., Shimizu, S., Ito, T., Wang, L., Creaney, J., Lake, R. A., Zakowski, M. F., Reva, B., et al. (2011). The nuclear deubiquitinase BAP1 is commonly inactivated by somatic mutations and 3p21.1 losses in malignant pleural mesothelioma. Nature genetics 43, 668-672.
Campbell, J. E., Kuntz, K. W., Knutson, S. K., Warholic, N. M., Keilhack, H., Wigle, T. J., Raimondi, A., Klaus, C. R., Rioux, N., Yokoi, A., et al. (2015). EPZ011989, A Potent, Orally-Available EZH2 Inhibitor with Robust in
Vivo Activity. ACS Mod Chem Lett 6, 491-495.
Dey, A., Seshasayee, D., Noubade, R., French, D. M., Liu, J., Chaurushiya, M. S., Kirkpatrick, D. S., Pham, V. C., Lill, J. R., Bakalarski, C. E., et al. (2012). Loss of the tumor suppressor BAP1 causes myeloid transformation. Science 337, 1541-1546.
Garcia, B. A., Mollah, S., Ueberheide, B. M., Busby, S. A., Muratore, T. L., Shabanowitz, J., and Hunt, D. F. (2007). Chemical derivatization of histones for facilitated analysis by mass spectrometry. Nat Protoc 2, 933-938.
Gelsi-Boyer, V., Trouplin, V., Adelaide, J., Bonansea, J., Cervera, N., Carbuccia, N., Lagarde, A., Prebet, T., Nezri, M., Sainty, D., et al. (2009). Mutations of polycomb-associated gene ASXL1 in myelodysplastic syndromes and chronic myelomonocytic leukaemia. British journal of haematology 145, 788-800.
Guo, Y., Nady, N., Qi, C., Allali-Hassani, A., Zhu, H., Pan, P., Adams-Cioaba, M. A., Amaya, M. F., Dong, A., Vedadi, M., et al. (2009). Methylation-state-specific recognition of histones by the MBT repeat protein L3MBTL2. Nucleic Acids Res 37, 2204-2210.
Harbour, J. W., Onken, M. D., Roberson, E. D., Duan, S., Cao, L., Worley, L. A., Council, M. L., Matatall, K. A., Helms, C., and Bowcock, A. M. (2010). Frequent mutation of BAP1 in metastasizing uveal melanomas. Science 330, 1410-1413.
Knutson, S. K., Warholic, N. M., Wigle, T. J., Klaus, C. R., Allain, C. J., Raimondi, A., Porter Scott, M., Chesworth, R., Moyer, M. P., Copeland, R. A, et al. (2013). Durable tumor regression in genetically altered malignant rhabdoid tumors by inhibition of methyltransferase EZH2. Proc Natl Acad Sci USA 110, 7922-7927.
Krivtsov, A. V., Feng, Z., Lemieux, M. E., Faber, J., Vempati, S., Sinha, A. U., Xia, X., Jesneck, J., Bracken, A. P., Silverman, L. B., et al. (2008). H3K79 methylation profiles define murine and human MLL-AF4 leukemias. Cancer Cell 14, 355-368.
Lara-Astiaso, D., et al. Immunogenetics. Chromatin state dynamics during blood formation. Science 345, 943-949 (2014).
Love, M. I., Huber, W., and Anders, S. (2014). Moderated estimation of fold change and dispersion for RNA-seq data with DESeq2. Genome biology 15, 550.
MacLean, B., Tomazela, D. M., Shulman, N., Chambers, M., Finney, G. L., Frewen, B., Kern, R., Tabb, D. L., Liebler, D. C., and MacCoss, M. J. (2010). Skyline: an open source document editor for creating and analyzing targeted proteomics experiments. Bioinformatics 26, 966-968.
McCabe, M. T., Ott, H. M., Ganji, G., Korenchuk, S., Thompson, C., Van Aller, G. S., Liu, Y., Graves, A. P., Della Pictra, A., 3rd, Diaz, E., et al. (2012). EZH2 inhibition as a therapeutic strategy for lymphoma with EZH2-activating mutations. Nature 492, 108-112.
Morin, R. D., Johnson, N. A., Severson, T. M., Mungall, A. J., An, J., Goya, R., Paul, J. E., Boyle, M., Woolcock, B. W., Kuchenbauer, F., et al. (2010). Somatic mutations altering EZH2 (Tyr641) in follicular and diffuse large B-cell lymphomas of germinal-center origin. Nature genetics 42, 181-185.

Morin, R. D., Mendez-Lago, M., Mungall, A. J., Goya, R., Mungall, K. L., Corbett, R. D., Johnson, N. A., Severson, T. M., Chiu, R., Field, M., et al. (2011). Frequent mutation of histone-modifying genes in non-Hodgkin lymphoma. Nature 476, 298-303.
Nishioka, K., Rice, J. C., Sarma, K., Erdjument-Bromage, H., Werner, J., Wang, Y., Chuikov, S., Valenzuela, P., Tempst, P., Steward, R., et al. (2002). PR-Set7 is a nucleosome-specific methyltransferase that modifies lysine 20 of histone H4 and is associated with silent chromatin. Mol Cell 9, 1201-1213.
O'Geen, H., Echipare, L., and Farnham, P. J. (2011). Using ChIP-seq technology to generate high-resolution profiles of histone modifications. Methods Mol Biol 791, 265-286.
Pasqualucci, L., Trifonov, V., Fabbri, G., Ma, J., Rossi, D., Chiarenza, A., Wells, V. A., Grunn, A., Messina, M., Elliot, O., et al. (2011). Analysis of the coding genome of diffuse large B-cell lymphoma. Nat Genet 43, 830-837.
Pena-Llopis, S., Vega-Rubin-de-Celis, S., Liao, A., Leng, N., Pavia-Jimenez, A., Wang. S., Yamasaki, T., Zhrebker, L., Sivanand, S., Spence, P., et al. (2012). BAP1 loss defines a new class of renal cell carcinoma. Nat Genet 44, 751-759.
Phung, Y. T., Barbone, D., Broaddus, V. C., and Ho, M. (2011). Rapid generation of in vitro multicellular spheroids for the study of monoclonal antibody therapy. Journal of Cancer 2, 507-514.
Qin, J., Van Buren, D., Huang, H. S., Zhong, L, Mostoslavsky, R., Akbarian, S., and Hock, H. (2010). Chromatin protein L3MBTL1 is dispensable for development and tumor suppression in mice. J Biol Chem 285, 27767-27775.
Qin, J., Whyte, W. A., Anderssen, E., Apostolou, E., Chen, H. H., Akbarian, S., Bronson, R. T., Hochedlinger, K., Ramaswamy, S., Young. R. A., et al. (2012). The polycomb group protein L3mbtl2 assembles an atypical PRC1-family complex that is essential in pluripotent stem cells and early development. Cell Stem Cell 11, 319-332.
Scheuermann, J. C., de Ayala Alonso, A. G., Oktaba, K., Ly-Hartig, N., McGinty, R. K., Fraterman, S., Wilm, M., Muir, T. W., and Muller, J. (2010). Histone H2A deubiquitinase activity of the Polycomb repressive complex PR-DUB. Nature 465, 243-247.
Skarnes, W. C., Rosen, B., West, A. P., Koutsourakis, M., Bushell, W., lyer, V., Mujica, A. O., Thomas, M., Harrow, J., Cox, T., et al. (2011). A conditional knockout resource for the genome-wide study of mouse gene function. Nature 474, 337-342.
Su, I. H., Basavaraj, A., Krutchinsky, A. N., Hobert, O., Ullrich, A., Chait, B. T., and Tarakhovsky, A. (2003). Ezh2 controls B cell development through histone H3 methylation and Igh rearrangement. Nat Immunol 4, 124-131.
Suraokar, M. B., Nunez, M. I., Diao, L., Chow, C. W., Kim, D., Behrens, C., Lin, H., Lee, S., Raso, G., Moran, C., et al. (2014). Expression profiling stratifies mesothelioma tumors and signifies deregulation of spindle checkpoint pathway and microtubule network with therapeutic implications. Annals of oncology: official journal of the European Society for Medical Oncology/ESMO 25, 1184-1192.
Trojer, P., Cao, A. R., Gao, Z., Li, Y., Zhang, J., Xu, X., Li, G., Losson, R., Erdjument-Bromage, H., Tempst, P., et al. (2011). L3MBTL2 protein acts in concert with PcG protein-mediated monoubiquitination of H2A to establish a repressive chromatin structure. Mol Cell 42, 438-450.
Trojer, P., Li, G., Sims, R. J., 3rd, Vaquero, A., Kalakonda, N., Boccuni, P., Lee, D., Erdjument-Bromage, H., Tempst, P., Nimer, S. D., et al. (2007). L3MBTL1, a histone-methylation-dependent chromatin lock. Cell 129, 915-928.
Wilson, B. G., Wang, X., Shen, X., McKenna, E. S., Lemieux, M. E., Cho, Y. J., Koellhoffer, E. C., Pomeroy, S. L., Orkin, S. H., and Roberts, C. W. (2010). Epigenetic antagonism between polycomb and SWI/SNF complexes during oncogenic transformation. Cancer Cell 18, 316-328.

Various references are cited herein, the contents of which are hereby incorporated by reference in their entireties. Various nucleic acid and amino acid sequence accession numbers are cited herein, and the complete sequences referenced by those accession numbers are hereby incorporated by reference in their entireties.

We claim:

1. A method for determining whether an anti-cancer effect is likely to be produced in a cancer by an EZH2 inhibitor, comprising determining the expression of a BAP1 biomarker in one or more cells of the cancer, where if the BAP1 biomarker is absent or expressed at lower levels in the cancer, as compared to a reference control level, then administering a therapeutically effective amount of an EZH2 inhibitor to produce an anti-cancer effect.

2. The method of claim 1, wherein the cancer is selected from the group consisting of malignant mesothelioma, uveal melanomas, renal cell carcinoma, cutaneous melanomas, lung cancer, breast cancer, ovarian cancer, non-melanoma skin cancer, meningioma, chlangiocarcinoma, leiomysarcoma, neuroendocrine tumors, pancreatic cancer, paraganglioma, malignant fibrous histiocytoma, melanocytic BAP1-mutated atypical intradermal tumors (MBAITs), acute myeloid leukemia, myelodysplastic syndromes and bladder cancer.

3. The method of claim 1, wherein the expression of the BAP1 biomarker is determined by immunofluorescence.

4. The method of claim 1, wherein the expression of the BAP1 biomarker is determined by Western Blot.

5. The method of claim 1, wherein the expression of the BAP1 biomarker is determined by in situ hybridization.

6. The method of claim 1, wherein the expression of the BAP1 biomarker is determined by polymerase chain reaction.

7. The method of claim 1, wherein the expression of the BAP1 biomarker is detected by using a reagent which specifically binds with the BAP1 biomarker.

8. The method of claim 1, wherein the reagent is an antibody or an antigen binding fragment thereof.

9. The method of claim 1, where the cancer is a malignant mesothelioma.

10. The method of claim 1, where the cancer is an uveal melanoma.

11. The method of claim 1, where the cancer is a renal cell carcinoma.

12. The method of claim 1, further comprising determining the expression level of EZH2 in the sample.

13. A method for treating a subject having a cancer, comprising, obtaining a sample of the cancer from the subject, and determining, in the sample, the expression level of a BAP1 biomarker and/or the expression level of EZH2 and/or SUZ12, where if the BAP1 biomarker is absent or expressed at a lower level than a BAP1 reference control level and/or if the expression of EZH2 and/or SUZ12 is increased compared to an EZH2 reference control level, then initiating treatment of the subject with a therapeutically effective amount of an EZH2 inhibitor.

14. The method of claim 13, where the cancer is a malignant mesothelioma.

15. The method of claim 13, where the cancer is an uveal melanoma.

16. The method of claim 13, where the cancer is a renal cell carcinoma.

17. The method of claim 13, wherein the expression of the BAP1 biomarker, SUZ12 and EZH2 is determined by immunofluorescence.

18. The method of claim 13, wherein the expression of the BAP1 biomarker, SUZ12 and EZH2 is determined by Western Blot.

19. A method for determining whether an anti-cancer effect is likely to be produced in a cancer by an EZH2 inhibitor, comprising obtaining a sample of the cancer from a subject, and determining, in the sample, the expression level of an BAP1 biomarker, where if the BAP1 biomarker is absent or expressed at lower level in the cancer, as compared to a reference control level, it is more likely that the EZH2 inhibitor would have an anti-cancer effect on the cancer.

20. The method of claim 19, wherein the cancer is selected from the group consisting of malignant mesotheliomas, uveal melanomas, renal cell carcinoma, cutaneous melanomas, lung cancer, breast cancer, ovarian cancer, non-melanoma skin cancer, meningioma, chlangiocarcinoma, leiomysarcoma, neuroendocrine tumors, pancreatic cancer, paraganglioma, malignant fibrous histiocytoma, melanocytic BAP1-mutated atypical intradermal tumors (MBAITs), acute myeloid leukemia, myelodysplastic syndromes and bladder cancer.

21. The method of claim 19, wherein the BAP1 biomarker is a BAP1 protein biomarker.

22. The method of claim 19, wherein the BAP1 biomarker is a BAP1 nucleic acid biomarker.

23. The method of claim 19, wherein the expression of the BAP1 biomarker is determined by immunofluorescence.

24. The method of claim 19, wherein the expression of the BAP1 biomarker is determined by Western Blot.

25. A method of predicting the sensitivity of a cancer in a patient to an EZH2 inhibitor, comprising, obtaining a sample of the cancer from the patient and determining the expression level of a BAP1 protein biomarker in the cells comprising the sample, wherein if the BAP1 biomarker is absent or reduced in expression level compared to a reference control level, then the cancer is predicted to be sensitive to an EZH2 inhibitor.

26. The method of claim 25, wherein the cancer is selected from the group consisting of malignant mesotheliomas, uveal melanomas, renal cell carcinoma, cutaneous melanomas, lung cancer, breast cancer, ovarian cancer, non-melanoma skin cancer, meningioma, chlangiocarcinoma, leiomysarcoma, neuroendocrine tumors, pancreatic cancer, paraganglioma, malignant fibrous histiocytoma, melanocytic BAP1-mutated atypical intradermal tumors (MBAITs), acute myeloid leukemia, myelodysplatic syndromes and bladder cancer.

27. A kit for determining whether an anti-cancer effect is likely to be produced in a cancer by an EZH2 inhibitor, comprising a means for detecting a BAP1 biomarker.

28. The kit of claim 27, wherein the means for detecting a BAP1 biomarker comprises one or more packaged primers, probe, arrays/microarray, biomarker-specific antibody and/or bead.

29. The kit of claim 27, wherein the means for detecting a BAP1 biomarker comprises one or more antibodies, or antigen binding fragment thereof, for detecting a BAP1 biomarker.

30. The kit of claim 27, wherein the kit further comprises one or more primers, probe, arrays/microarray, biomarker-specific antibody and/or bead for detecting EZH2 expression, L3MBTL2 expression and/or SUZ12 expression.


SEQUENCE LISTING (41 sequences)

SEQ ID NO: 1 - DNA, 20 bp, Artificial Sequence
  Description of Artificial Sequence Synthetic primer
actgcagcaatgtggatctg

SEQ ID NO: 2 - DNA, 21 bp, Artificial Sequence
  Description of Artificial Sequence Synthetic primer
gaaaaggtctgacccagatca

SEQ ID NO: 3 - DNA, 20 bp, Artificial Sequence
  Description of Artificial Sequence Synthetic primer
gcgcaacgcaattaatgata

SEQ ID NO: 4 - DNA, 20 bp, Artificial Sequence
  Description of Artificial Sequence Synthetic primer
cagtgtccagaatggctcaa

SEQ ID NO: 5 - DNA, 36 bp, Artificial Sequence
  Description of Artificial Sequence Synthetic primer
ccaccagctgatacccaactctgctgcaactcatgc

SEQ ID NO: 6 - DNA, 36 bp, Artificial Sequence
  Description of Artificial Sequence Synthetic primer
gcatgagttgcagcagagttgggtatcagctggtgg

SEQ ID NO: 7 - DNA, 22 bp, Artificial Sequence
  Description of Artificial Sequence Synthetic primer
agcacaagtcatcccgttaaag

SEQ ID NO: 8 - DNA, 21 bp, Artificial Sequence
  Description of Artificial Sequence Synthetic primer
aattctgttgtaagggcgacc

SEQ ID NO: 9 - DNA, 19 bp, Artificial Sequence
  Description of Artificial Sequence Synthetic primer
ggctgaccacgagcttttc

SEQ ID NO: 10 - DNA, 23 bp, Artificial Sequence
  Description of Artificial Sequence Synthetic primer
tggtgcgataagatttcgagttc

SEQ ID NO: 11 - DNA, 20 bp, Artificial Sequence
  Description of Artificial Sequence Synthetic primer
gttggtggatgacacgtctg

SEQ ID NO: 12 - DNA, 20 bp, Artificial Sequence
  Description of Artificial Sequence Synthetic primer
ctcaggactgaagcctttgg

SEQ ID NO: 13 - DNA, 20 bp, Artificial Sequence
  Description of Artificial Sequence Synthetic primer
gatctggcaccacaccttct

SEQ ID NO: 14 - DNA, 20 bp, Artificial Sequence
  Description of Artificial Sequence Synthetic primer
ccatcacaatgcctgtggta

SEQ ID NO: 15 - DNA, 19 bp, Artificial Sequence
  Description of Artificial Sequence Synthetic primer
gctcagccccagatctacc

SEQ ID NO: 16 - DNA, 20 bp, Artificial Sequence
  Description of Artificial Sequence Synthetic primer
ggcatgagctatttcgatcc

SEQ ID NO: 17 - DNA, 19 bp, Artificial Sequence
  Description of Artificial Sequence Synthetic primer
ccctgtttacccctggatg

SEQ ID NO: 18 - DNA, 20 bp, Artificial Sequence
  Description of Artificial Sequence Synthetic primer
accgaccggaagtacacaag

SEQ ID NO: 19 - DNA, 20 bp, Artificial Sequence
  Description of Artificial Sequence Synthetic primer
cttctccagttccagcgtct

SEQ ID NO: 20 - DNA, 20 bp, Artificial Sequence
  Description of Artificial Sequence Synthetic primer
aggtagcggttgaaatggaa

SEQ ID NO: 21 - DNA, 20 bp, Artificial Sequence
  Description of Artificial Sequence Synthetic primer
atgcttgtggttctcctcca

SEQ ID NO: 22 - DNA, 20 bp, Artificial Sequence
  Description of Artificial Sequence Synthetic primer
gttccagcgtctggtgtttt

SEQ ID NO: 23 - DNA, 19 bp, Artificial Sequence
  Description of Artificial Sequence Synthetic primer
gaccggtgcaatcttcaaa

SEQ ID NO: 24 - DNA, 19 bp, Artificial Sequence
  Description of Artificial Sequence Synthetic primer
ttgacgccgagagctacac

SEQ ID NO: 25 - DNA, 19 bp, Artificial Sequence
  Description of Artificial Sequence Synthetic primer
cattatgccgaggatttgg

SEQ ID NO: 26 - DNA, 19 bp, Artificial Sequence
  Description of Artificial Sequence Synthetic primer
gcaagtctttcagtcctgt

SEQ ID NO: 27 - DNA, 21 bp, Artificial Sequence
  Description of Artificial Sequence Synthetic primer
cgatccatttgaacaggaaga

SEQ ID NO: 28 - DNA, 20 bp, Artificial Sequence
  Description of Artificial Sequence Synthetic primer
ctcgtggaagatttcggtgt

SEQ ID NO: 29 - DNA, 20 bp, Artificial Sequence
  Description of Artificial Sequence Synthetic primer
agctgactcaagctgcttgt

SEQ ID NO: 30 - DNA, 20 bp, Artificial Sequence
  Description of Artificial Sequence Synthetic primer
caggaaacctgagattttca

SEQ ID NO: 31 - DNA, 20 bp, Artificial Sequence
  Description of Artificial Sequence Synthetic primer
ctcaggacagttctgtttgg

SEQ ID NO: 32 - DNA, 20 bp, Artificial Sequence
  Description of Artificial Sequence Synthetic primer
tctgacttagttggagaact

SEQ ID NO: 33 - DNA, 27 bp, Artificial Sequence
  Description of Artificial Sequence Synthetic primer
tgaatacagatgcagttataagagaga

SEQ ID NO: 34 - DNA, 21 bp, Artificial Sequence
  Description of Artificial Sequence Synthetic primer
catcttccccaagctcccaat

SEQ ID NO: 35 - DNA, 21 bp, Artificial Sequence
  Description of Artificial Sequence Synthetic primer
gagcgagctacaaagccagga

SEQ ID NO: 36 - DNA, 20 bp, Artificial Sequence
  Description of Artificial Sequence Synthetic primer
cctgttcccttcctctggaa

SEQ ID NO: 37 - DNA, 20 bp, Artificial Sequence
  Description of Artificial Sequence Synthetic primer
cgacgcagacggaaaaagag

SEQ ID NO: 38 - DNA, 20 bp, Artificial Sequence
  Description of Artificial Sequence Synthetic primer
tgagtggggacttcgtgttc

SEQ ID NO: 39 - DNA, 20 bp, Artificial Sequence
  Description of Artificial Sequence Synthetic primer
gaccaaccgacagaaggact

SEQ ID NO: 40 - DNA, 20 bp, Artificial Sequence
  Description of Artificial Sequence Synthetic primer
tccacccctaggctgaaaag

SEQ ID NO: 41 - DNA, 20 bp, Artificial Sequence
  Description of Artificial Sequence Synthetic primer
gatcggctttgtgtctggtc